SEQUENCE LISTING (10 sequences)

SEQ ID NO: 1 - protein, 10 aa, synthetic construct
GGGGSGGGGS

SEQ ID NO: 2 - RNA, 21 nt, synthetic construct
  source 1..21: mol_type = other RNA
cgaagacgtttgtgggtgatt

SEQ ID NO: 3 - RNA, 21 nt, synthetic construct
  source 1..21: mol_type = other RNA
gcctatgttcgggacttcaaa

SEQ ID NO: 4 - RNA, 21 nt, synthetic construct
  source 1..21: mol_type = other RNA
gcagcagtttaaccagagatt

SEQ ID NO: 5 - RNA, 21 nt, synthetic construct
  source 1..21: mol_type = other RNA
aatccagaccacctgaaatac

SEQ ID NO: 6 - RNA, 21 nt, synthetic construct
  source 1..21: mol_type = other RNA
cctaaggttaagtcgccctcg

SEQ ID NO: 7 - DNA, 23 bp, synthetic construct
  source 1..23: mol_type = other DNA
tgccgactacaagcgaattactg

SEQ ID NO: 8 - DNA, 22 bp, synthetic construct
  source 1..22: mol_type = other DNA
ctgcttgtccagatgacttcgg

SEQ ID NO: 9 - DNA, 22 bp, synthetic construct
  source 1..22: mol_type = other DNA
gcacgatggattcggttaggtc

SEQ ID NO: 10 - DNA, 21 bp, synthetic construct
  source 1..21: mol_type = other DNA
tcggctccctctgtgtagtgg

(19) United States
(12) Patent Application Publication
(10) Pub. No.: US 2025/0101108 A1
(43) Pub. Date: Mar. 27, 2025

(54) METHODS TO ENHANCE THERAPEUTIC EFFICACY IN MELANOMA VIA MODULATION OF CELL SURFACE PD-L1/L2

(71) Applicant: THE REGENTS OF THE UNIVERSITY OF CALIFORNIA, OAKLAND, CA (US)

(72) Inventors: Roger S. Lo, LOS ANGELES, CA (US); Zhentao Yang, LOS ANGELES, CA (US)

(73) Assignee: THE REGENTS OF THE UNIVERSITY OF CALIFORNIA, OAKLAND, CA (US)

(21) Appl. No.: 18/709,427

(22) PCT Filed: Nov. 16, 2022
(86) PCT No.: PCT/US2022/079966

§ 371 Date: May 10, 2024

Related U.S. Application Data
(60) Provisional application No. 63/364,010, filed on May 2, 2022.
(60) Provisional application No. 63/265,237, filed on Dec. 10, 2021.
(60) Provisional application No. 63/264,130, filed on Nov. 16, 2021.

(51) Int. Cl. C07K 16/28; A61K 31/18; A61K 31/352; A61K 31/409; A61K 31/4162; A61K 31/454; A61K 31/519; A61K 39/00; A61K 45/06; A61P 35/00

(52) U.S. Cl. CPC C07K 16/2827; A61K 31/18; A61K 31/352; A61K 31/409; A61K 31/4162; A61K 31/454; A61K 31/519; A61K 45/06; A61P 35/00; A61K 2039/505

(57) ABSTRACT

Described are methods for enhancing cancer immune surveillance and the efficacy of the treatment of melanoma or non-melanoma cancers such as pancreatic or colorectal cancers, particularly by controlling cancer cell-surface PD-L1/L2 and the E3 ligase ITCH. Also described are methods of inhibiting an adaptive immune resistance response to anti-immune checkpoint protein therapy in a subject. As described herein, the efficacy of MAPK inhibitor therapy is increased by treatment with an activator of E3 ligase ITCH or a destabilizer of cell-surface PD-L1/L2. As further demonstrated herein, the efficacy of anti-PD-1/L1 therapy is also increased by treatment with an activator of E3 ligase ITCH or a destabilizer of cell-surface PD-L1/L2.

This application claims benefit of United States provisional patent application numbers 63/264,130, filed Nov. 16, 2021, 63/265,237, filed Dec. 10, 2021, and 63/364,010, filed on May 2, 2022, the entire contents of each of which are incorporated by reference into this application.

### STATEMENT REGARDING FEDERALLY SPONSORED RESEARCH

This invention was made with government support under Grant Numbers CA176111, CA255837, and CA168585, awarded by the National Institutes of Health. The government has certain rights in the invention.

### REFERENCE TO A SEQUENCE LISTING SUBMITTED VIA EFS-WEB

The content of the XML file of the sequence listing named “UCLA285_seq”, which is 10 kb in size, created on Nov. 14, 2022, and electronically submitted herewith the application, is incorporated herein by reference in its entirety.

### BACKGROUND

Although MAPK- and PD-1/L1-targeted therapies first appeared to be therapies based on disparate mechanisms, we and others have identified shared and cross-synergistic immunologic mechanisms of action. For example, in clinical melanoma, acquired resistance to MAPK-targeted therapy frequently involves immune evasion and, in murine melanoma, anti-melanoma CD8+ T cells are critical for the depth and durability of MAPK inhibitor-elicited responses. Recently, we showed that the benefits of rational sequencing plus combination of MAPK- and PD-1/L1-targeted therapies are derived from immunologic mechanisms. The clinical implication is that their rational sequencing-combination can help reduce resistance to either therapy. Although MAPK-targeted therapy (centered on BRAFV600 mutations) was first developed in melanoma, the only regimens approved over the last decade to suppress resistance have been the combination of BRAF^{V600MUT} plus MEK inhibitors and the more recent combination of BRAF^{V600MUT}, MEK, and PD-L1 inhibitors.

Disrupting PD-L1 interaction with PD-1 rejuvenates antitumor immunity and elicits clinical antitumor responses across a wide-range of cancer histologies. Cancer cells can express robust surface levels of PD-L1 to tolerize tumor-specific T cells, but regulation of PD-L1 protein levels on the tumor cell surface is poorly understood. De-differentiated or quasi-mesenchymal tumor cells up-regulate PD-L1/L2 (or vice versa) and induce an immune-suppressive microenvironment, including expansion of M2-like macrophages and regulatory T cells as well as depletion of CD8+ T cells. Targeted therapy, including MAPKi therapy in melanoma, leads to de-differentiation, PD-L1/2 upregulation, and resistance, and both MAPKi treatment and mesenchymal signatures are associated with innate anti-PD-1 resistance.

There remains a need for improving the efficacy of melanoma therapies, particularly for reducing immune evasion by modulation of tumor cell-surface PD-L1.

### SUMMARY

The methods described herein meet these needs and more by providing compositions and methods for enhancing cancer immune surveillance and the efficacy of the treatment of cancers such as melanoma, pancreatic ductal adenocarcinoma, and colorectal adenocarcinoma, particularly by controlling cancer cell-surface PD-L1/L2 and the E3 ligase ITCH. In some embodiments, described herein is a method of suppressing tumor cell-surface PD-L1 expression to thereby enhance anti-cancer therapy in a subject in need thereof. In some embodiments, the method comprises administering to the subject an effective amount of a modulator of tumor cell-surface PD-L1/L2. In some embodiments, the modulator of tumor cell-surface PD-L1/L2 is an activator of E3 ligase ITCH and/or a destabilizer of tumor cell-surface PD-L1/L2. In some embodiments, the cancer is melanoma. In some embodiments, the cancer is pancreatic ductal adenocarcinoma. In some embodiments, the cancer is colorectal adenocarcinoma.

In some embodiments, the activator of ITCH is selected from: Chlorophyllide, 3-chloro-1-(4-methylphenyl)-4-piperidin-1-yl-1H-pyrrole-2,5-dione, N-(3,4-dimethylphenyl)benzenesulfonamide, 4-methylphenyl 7-methylpyrrolo[1,2-c]pyrimidin-3-yl sulfone, 5,6,7-trimethoxy-4-methyl-2H-chromen-2-one (AK087), and 6-amino-3-methyl-4-{2-[(1-methylethyl)oxy]phenyl}-2,4-dihydropyrano[2,3-c]pyrazole-5-carbonitrile. In some embodiments, the activator of ITCH is 5,6,7-trimethoxy-4-methyl-2H-chromen-2-one (AK087). In some embodiments, the destabilizer of cell-surface PD-L1/L2 is a recombinant bispecific antibody-based proteolysis-targeting chimera (AbTAC) that recruits membrane-bound E3 ligases.

In some embodiments, the subject is treated with one or more mitogen-activated protein kinase (MAPK) inhibitors. In some embodiments, the subject is treated with one or more anti-PD-1/L1 antibodies, and/or with one or more antibodies to other immune checkpoint proteins, as anti-cancer therapy, for example, such as anti-melanoma therapy. Examples of antibodies to other immune checkpoint proteins include, but are not limited to, anti-CTLA-4 antibodies, anti-PD-1 antibodies, anti-PD-L1 antibodies, and/or anti-LAG-3 antibodies. In some embodiments, the subject is treated with both MAPK inhibitor(s) and anti-PD-1/L1 antibodies (and/or antibodies to other immune checkpoint proteins). In some embodiments, the MAPK inhibitor(s), or MAPK inhibitor(s) plus anti-PD-1/L1/anti-immune checkpoint protein antibodies, is administered concomitantly with, prior to, and/or subsequent to the administering of the activator of ITCH or destabilizer of PD-L1/L2. In some embodiments, the MAPK inhibitor is selected from: Vemurafenib, Dabrafenib. Encorafenib, Trametinib, Binimetinib, and Cobimetinib, as well as type II RAF inhibitors or pan-RAF inhibitors, such as BGB-283, BGB-3245, DAY101/TAK-580, KIN-2787, and LXH254.

Also described herein is a method of inhibiting an adaptive immune resistance response to anti-PD-1/L1 therapy, or other immune checkpoint therapy, in a subject in need thereof. In some embodiments, the method comprises administering to the subject an effective amount of an activator of E3 ligase ITCH or a destabilizer of cell-surface PD-L1/L2. In some embodiments, the activator of E3 ligase ITCH or destabilizer of cell-surface PD-L1/L2 is 5,6,7-trimethoxy-4-methyl-2H-chromen-2-one (AK087). In some embodiments, the immune checkpoint therapy comprises anti-CTLA-4 antibody, anti-PD-1 antibody, anti-PD-L1 antibody, and/or anti-LAG-3 antibody treatment.

### BRIEF DESCRIPTION OF THE DRAWINGS

FIGS. 1A-1H demonstrate that the E3 ligase ITCH interacts with PD-L1 and PD-L2. (1A) Spectral count of indicated proteins from shotgun mass spectrometry-based analysis of anti-FLAG immunoprecipitates from the M229 R5 cell line engineered to express PD-L2-FLAG (n=2) or empty vector (control, n=4). Average total spectral count in samples: 5,156 (control), 4,229 (anti-FLAG), mean±SEMs. Proteins shown with confidence score for interaction (SAINT, Significance Analysis of INTeractome) higher than 0.95. (1B) Proteins identified in proximity to PD-L1 in M229 R5 cell line stably expressing PD-L1-APEX2 (H₂O₂ treated vs. not treated, n=3). Black dots, proteins significantly enriched in H₂O₂-treated cell. (1C) Immunoprecipitation (IP) of HEK 293T cells transfected with PD-L1 and ITCH-FLAG (alone or together) using anti-FLAG M2 beads followed by Western blots (WB) of indicated proteins. TUBULIN, loading control. (1D) IP of HEK 293T cells transfected with PD-L2-FLAG and/or ITCH-HA using anti-HA beads followed by WBs of indicated proteins. (1E) IP of the human melanoma cell line M238 R1 stably expressing ITCH using anti-PD-L1 antibody (rabbit-IgG as control) followed by Western blotting of indicated proteins. (1F) IP of the human non-small cell lung carcinoma cell line H358, with or without ITCH overexpression (OE), using anti-PD-L1 (rabbit-IgG as control) followed by Western blotting of indicated proteins. (1G) PD-L1 protein levels compared by splitting 7,194 patient-derived tumors with matched RNA-seq data and PD-L1 protein levels (measured by reverse-phase protein array from 32 TCGA cancer types) into top versus bottom 20% of ITCH RNA expression levels. (1H) Kaplan-Meier survival curve of 471 cutaneous melanoma patients from TCGA dataset with high (top 20%) versus low (bottom 20%) ITCH RNA expression levels. Time of diagnosis set as the starting time for follow-up. HR, hazard ratio

FIGS. 2A-2N show that ITCH poly-ubiquitinates PD-L1 and promotes T-cell activation by down-regulating tumor cell-surface levels of PD-L1. FIGS. 2A, 2B, and 2C, Western blots (WBs) of M238 R1 (2A), H358 (2B), or MDA-MB-231 (2C) cells stably expressing control shRNA (shCtr) or ITCH-targeting shRNAs (shiICH-1, shiICH-2) (left). Cell-surface levels of PD-L1 measured by cell-surface staining and flow cytometry analysis (right). MFI, mean fluorescent intensity. Mean±SEMs (n=3). P value, Student's t test, ***p<0.001. (2D) (left) WBs of M238 R1 cells stably expressing empty vector (Vec) or over-expressing (OE) ITCH. (right) Cell-surface levels of PD-L1 measured by cell-surface staining and FACS analysis. Mean±SEMs (n=3). P value, Student t test, ***p<0.001. (2E) HEK 293T cells transiently expressing PD-L1-FLAG, with or without ITCH co-transfection, were pretreated with or without MG-132 (20 μM) for 4 hours followed by anti-FLAG IP and detection of ubiquitin (UB) by WBs. (2F) M238 R1 or (2G) H358 cells expressing shCtr or ITCH-shRNAs were pretreated with MG-132 (20 μM) for 4 hours followed by anti-PD-L1 IP and UB detection by WBs. (2H) M238R1 cells expressing Vec or OE ITCH were pretreated with MG-132 (20 μM) for 4 hours followed by anti-PD-L1 IP and then WB detection of UB. (2I) Schematic of co-culture assay used in 2J to 2L. (2J), (2K) WBs of indicated proteins in NYESO-HLA-A2-expressing M238 R1 (2J) or H358 (2K) cells with shCtr or ITCH-shRNAs stable expression (left). IL-2 production measured by ELISA assay after co-culture experiments with indicated cell lines (right). Jk, Jurkat T cells. Mean±SEMs (n=3). P values, Student's t test, **p<0.01, ***p<0.001. (2L) IL-2 production measured by ELISA assay after co-culture experiment with indicated cell lines and treatment. Anti-PD-1 and anti-PD-1 antibodies were added in the culture medium at the final concentration of 10 mg/mL (each antibody) and pre-incubated with Jk T cells for 30 mins before co-culture with target cells. Mean±SEMs (n=4). P value, Student's t test, ***p<0.001. (2M), (2N) IL-2 production measured by ELISA assay after co-culture of human peripheral blood mononuclear cells (hPBMCs) with M238R1 (2M) or H358 (2N) cell lines. Anti-CD3 and anti-CD28 antibodies were added at the final concentration of 1 mg/mL (each antibody) to activate hPBMCs. Anti-PD-1 and anti-PD-L1 antibodies was added at the final concentration of 10 mg/ml (each antibody). Mean±SEMs (n=4). P values, Student's t test, ***p<0.001.

FIGS. 3A-3H show that ITCH limits MAPKi-elicited accumulation of tumor cell-surface PD-L1 and suppresses resistance only in immune-competent hosts. (3A) FACS analysis of YUMM1.7ER tumor surface PD-L1/L2 levels, with or without seven days of trametinib (1 mg/kg/d) treatment (Tram D7, no treatment or NT D7). Subcutaneously growing tumors were dissociated into single cells. Tumor cells were stained and gated as the CD45/CD90 double-negative population. See FIG. 10 for an example of gating strategy. Mean±SEMs (n=3). (3B) WBs of YUMM1.7ER cells stably expressing control or ITCH-shRNA (left). FACS analysis of tumor surface PD-L1 from shCONTROL (shCtr) and ITCH-knockdown (shiICH-3) YUMM1.7ER tumors under NT on DO or Tram on D7 (1 mg/kg/d) (right). Average PD-L1 expression of the CD45⁺ population was used as an internal control to normalize measurements of tumor surface PD-L1 levels on different days. Mean±SEMs (n=4 for NT DO groups and n=3 for Tram D7 groups). (3C) Growth curves of shCtr and shiICH-3 YUMM1.7ER tumors under NT or Tram (1 mg/kg/d) treatment in C57BL/6 mice. Mean #SEMs (n=6 for NT groups and n=8 for Tram groups). (3D) Growth curves of shCtr and ITCH-knockdown (shiICH-mix) YUMM1.7ER tumors under BRAF^{V600MUT} inhibitor vemurafenib (Vem)+Tram treatment or Vem+Tram+anti-PD-L1 (Vem, 50 mg/kg/d; Tram, 0.3 mg/kg/d) in C57BL/6 mice. Mean±SEMs (n=7 for Vem+Tram groups and n=9 for Vem+Tram+anti-PD-L1 groups). shiICH-mix, admixture of lentiviruses of shiICH-2 and shiICH-3 to achieve higher knockdown efficiency. (3E) Growth curves of shCtr and shiICH-mix NILER1-4 tumors on NT or Tram (3 mg/kg/d) treatment in C57BL/6 mice (left or middle). WBs of the stable cell lines used for tumor engraftment (right). Mean±SEMs (n=6 for NT groups and n=10 for Tram groups). (3F), (3G) Growth curves of shCtr and shiICH-mix YUMM1.7ER (3F) or NILER 1-4 (3G) tumors on NT or Tram (1 mg/kg/d or 3 mg/kg/d) treatment in immune-deficient NSG mice (left or middle). WBs of the stable cell lines used for tumor engraftment (right). Mean #SEMs (n=7 for NT groups and n=8 for Tram groups). (3H) Growth curves of shCtr and shiICH-mix YUMM1.7ER tumors on NT or anti-PD-L1 treatment in C57BL/6 mice. Treatment started at tumor size ˜50 mm³. Mean±SEMs (n=6 for NT groups and n=8 for anti-PD-L1 groups). P value, Student's t-test, *p<0.05, **p<0.01, ***p<0.001, ns or not significant.

FIGS. 4A-4R demonstrate immune impacts of tumor-intrinsic ITCH entail CD8⁺ T cell-mediated suppression of MAPKi-resistance. (4A) t-Distribution Stochastic Neighbor Embedding (t-SNE) map of intratumoral CD4⁺ T cells from shCONTROL and ITCH-knockdown tumors on trametinib treatment. YUMMER1.7 tumors analyzed by CyTOF. Inferred cell types indicated by clusters with distinct colors. (4B) Fractions of indicated cell types in total CD4⁺ T cells from shCONTROL and ITCH-knockdown tumors (YUMM1.7ER), both on trametinib treatment. Mean±SEMs (n=3). P value, Student's t test. *p<0.05, **p<0.01, ***p<0.001. (4C) As in 4A, except intratumoral CD8⁺ T cells. (4D) As in 4B, except total CD8⁺ T cells. (4E) As in 4A, except n=4 (NILER1-4). (4F) As in 4B, except n=4 (NILER1-4). (4G) Spearman's correlation score (Rho) between intratumoral PD-L1 expression and TREG infiltration calculated by three different algorithms (CIBERSORT-ABS. QUANTISEQ and XCELL). Rho >0, positive correlation. P values, as indicated. (4H) As in 4A, except intratumoral CD8⁺ T cells from NILER1-4 tumors (n=4). (4I) As in 4B, except total CD8⁺ T cells and n=4 (NILER1-4). (4J) Uniform manifold approximation and projection (UMAP) of intratumoral T cells (n=8,405) analyzed by scRNA-seq (shCONTROL and ITCH-knockdown NILER1-4 tumors on trametinib treatment). Different cell clusters denoted by distinct colors. (4K) Fractions of indicated cell types in total T cells from shCONTROL and ITCH-knockdown NILER1-4 tumors. (4L) Heatmap showing expression levels of differentially expressed genes (rows) among different T-cell subpopulations (columns) in NILER1-4 tumors. Representative genes of each cluster are highlighted. (4M) UMAP in 4J colored by clonality based on scTCR-seq analysis (upper panel). Clonal expansion indices of T-cell subpopulations (lower panel) in NILER1-4 tumors. (4N) Transition indices between indicated CD8⁺ T-cell subpopulations in NILER1-4 tumors. (4O) UMAP of intratumoral TAMs (n=991) in NILER1-4 tumors analyzed by scRNA-seq. Different cell clusters denoted by distinct colors. (4P) As in 4I, except TAM subpopulations in NILER1-4 tumors. (4Q) Fractions of each TAM subpopulation in total CD45⁺ cells from shCONTROL and ITCH-knockdown NILER1-4 tumors, both on trametinib treatment. (4R) The ratio of M2-like TAMs to M1-like TAMs in shCONTROL and ITCH-knockdown NILER1-4 tumors, both on trametinib treatment.

FIGS. 5A-5M show that ITCH suppresses MAPKi-resistance by PD-L1 down-regulation and CD8⁺ T-cell up-regulation. (5A) FACS analysis of YUMM1.7ER (Vector-only or Vec; ITCH over-expression or OE) tumor-surface PD-L1 levels, with or without seven days of trametinib (0.45 mg/kg/d) treatment (Tram D7, no treatment or NT). Subcutaneously growing tumors were dissociated into single cells. Tumor cells were stained and gated as the CD45/CD90 double-negative population. Mean±SEMs (n=4). P value, Student's t test. ***p<0.001. (5B) Growth curves of Vec and ITCH OE YUMM1.7ER tumors on NT or trametinib (Tram, 0.45 mg/kg/d) treatment in C57BL/6 mice (left, middle). Mean±SEMs (n=8 for NT and n=10 for Tram groups). Western blots (WBs) of the stable cell lines used for tumor engraftment (right). (5C) Growth curves of Vec and ITCH OE NILER1-4 tumors on NT or trametinib (Tram, 3 mg/kg/d), alone or in combination with anti-CD8 (200 μg per mouse twice weekly) treatment, in C57BL/6 mice (left, middle). Mean±SEMs (n=7-10). WBs of the stable cell lines used for tumor engraftment (right). (5D) Growth curves of Vec and ITCH OE mSK-Mel254 tumors on NT or trametinib (Tram, 3 mg/kg/d) treatment in C57BL/6 mice (left). Mean±SEMs (n=8-10). WBs of the stable cell lines used for tumor engraftment (right). (5E) Growth curves of Vec and ITCH OE KPC tumors on NT or trametinib (Tram, 3 mg/kg/d) treatment in C57BL/6 mice (left). Mean±SEMs (n=10). WBs of the stable cell lines used for tumor engraftment (right). (5F) Growth curves of Vec and ITCH OE YUMM1.7ER tumors on trametinib (Tram, 0.45 mg/kg/d), alone or in combination with anti-CD8 (200 μg per mouse twice weekly) or RP832c (10 mg/kg/d, do to d7) treatment, in C57BL/6 mice. Mean±SEMs (n=8 for Tram and n=10 for Tram+anti-CD8 and Tram+RP832c groups). (5G) YUMM1.7ER Vec and ITCH OE stable lines were engineered to express Vec or PD-L1 (PD-L1 OE). WBs (left) and FACS analysis (right) of the total and cell-surface PD-L1 of the indicated doubly-transduced stable lines. (5H) Growth curves of engrafted tumors (derived from stable lines in 5G) on NT or trametinib (Tram, 0.45 mg/kg/d) treatment in C57BL/6 mice. Mean±SEMs (n=7-10). P value, Student's t test. *p<0.05, **p<0.01, ***p<0.001. (5I) t-SNE map of intratumoral CD45⁺ cells from Vec and ITCH-OE YUMMER1.7 tumors on NT and trametinib treatment and analyzed by CyTOF. Inferred cell types denoted by distinct colors. (5J) Fractions of indicated cell types in CD45⁺ cells from Vec and ITCH-OE YUMMER1.7 tumors on NT and trametinib treatment. Mean±SEMs (n=4). P value, Student's t test. *p<0.05, **p<0.01. (5K) As in 5I except for CD8⁺ T cells. (5L) As in 5J except for CD8⁺ T cells. (5M) Heatmap showing scaled mean expression levels of indicated protein markers in different cell clusters of CD8⁺ T cells in YUMM1.7ER tumors.

FIGS. 6A-6K show that AK087 is an ITCH activator that down-regulates tumor cell-surface PD-L1/L2 and suppresses MAPKi-resistance in vivo. (6A) Structure and chemical name of AK087. (6B) M238R1 cells with ITCH-FLAG over-expression were treated with 0, 40, or 80 μM of AK087 for 6 days and then treated with MG-132 (20 μM) for 4 hours followed by anti-PD-L1 or anti-FLAG IP and Western blot (WB) detection of UB. (6C) 293T cells co-transfected with PD-L1-FLAG and ITCH-HA were treated with 0, 40, or 80 μM of AK087 for 6 days and then treated with MG-132 (20 μM) for 4 hours followed by anti-HA or anti-FLAG IP and WB detection of UB. (6D) M238R1 cell-surface levels of PD-L1 (left) and PD-L2 (right) as measured by cell-surface staining and FACS analysis after 0, 20, 40, 80 μM AK087 treatment for 6 days. Mean±SEMs (n=3). P value, Student (test, ***p<0.001. (6E) As in 6D, except for H358 (PD-L2 is undetectable in H358). (6F), (6G) WBs of total PD-L1 protein levels in M238R1 (6F) and H358 (6G) cells after 6 days of treatment of vehicle (V or DMSO) or 20, 40, or 80 μM of AK087 (AK). (6H) Growth curves of YUMM1.7ER tumors on trametinib (Tram, 0.45 mg/kg/d) in combination with daily vehicle (4% Tween80+8% DMSO in double-distilled water) or AK087 (10 mg/kg/d, from d0 to d17) treatments in C57BL/6 mice. Mean±SEMs (n=9). CR, complete response. (6I) Weekly body weights of mice in 6H. (6J) Growth curves of NILER1-4 tumors on daily vehicle (4% Tween80+8% DMSO in double-distilled water), AK087 (10 mg/kg/day, from DO-D9), Tram (2 mg/kg/day) in combination with daily vehicle or AK087 (10 mg/kg/day, from DO-D18) treatment in C57BL/6 mice. Mean±SEMs (n=9-10). (6I) Weekly body weights of mice in 6J. P values, Student t test. *, P<0.05; **, P<0.01; ***, P<0.001.

FIG. 7 is a schematic illustration of a combinatorial strategy to reduce immune-mediated MAPKi-resistance. MAPK inhibitor (MAPKi) therapy of melanoma elicits tumor cell-surface PD-L1/L2 accumulation, which evades tumor antigen-specific cytolytic CD8⁺ T cells and potentially alters the phenotype or differentiation of intra-tumoral immune cell types such as TREG and tumor-associated macrophages (TAMs). This immune evasion or immune-suppressive tumor microenvironment reduces the durability of MAPKi responses, especially in tumors with high mutational or neoantigen burdens. ITCH, as an E3 ligase that ubiquitinates tumor cell-surface PD-L1/L2 and targets them for internalization and lysosomal degradation, can be activated pharmacologically during the early-phase of MAPKi therapy to enhance tumor rejection by cytolytic CD8⁺ T cells. Subsequent immunologic memory may suppress acquired MAPKi-resistance driven by non-immune or genetic mechanisms. Strategies alternative to ITCH activation may involve proteolysis targeting chimeras against PD-L1/L2 or depletion of TREG cells or M2-like TAMs.

FIGS. 8A-8E show correlations between tumoral ITCH RNA expression and PD-L1 protein levels, CD8⁺ T-cell infiltration, or patient survival. (8A) PD-L1 protein levels compared by splitting 7,194 patient-derived tumors with matched RNA-seq data and PD-L1 protein levels (measured by reverse-phase protein array from 32 TCGA cancer types) into top versus bottom 50% of ITCH RNA expression levels. (8B) Spearman's correlation score (Rho) between intra-tumoral ITCH RNA levels and PD-L1 protein levels in 7,194 tumors of 32 TCGA cancer types. Rho=−0.051, negative correlation. P=1.429e-05. (8C) Spearman's correlation score (Rho) between intra-tumoral ITCH RNA levels and CD8⁺ T-cell infiltration levels in TCGA-SKCM dataset (n=471) calculated by three different algorithms (CIBERSORT-ABS, EPIC and TIMER). Rho >0, positive correlation. P values, as indicated. (8D) Kaplan-Meier survival curve of stage-matched patients from TCGA-SKCM dataset with high (top 10%) versus low (bottom 10%) intra-tumoral ITCH expression (early stage: stage 0, I, II combined, n=231; left panel) or high (top 15%) versus low (bottom 15%) intra-tumoral ITCH expression (late stage: stage III, IV combined, n=193; right panel). Time of diagnosis as starting time for follow-up. P values, log-rank test. (8E) Kaplan-Meier survival curve of patients from TCGA renal clear cell carcinoma dataset with high (top 50%) versus low (bottom 50%) intra-tumoral ITCH expression. Time of diagnosis as starting time for follow-up. P values, log-rank test. HR, hazard ratio.

FIGS. 9A-9N show physical and functional interactions between ITCH and PD-L1/L2. (9A) Cell-surface levels of PD-L2 in M238R1 (left) and MDA-MB-231 (right) cells stably expressing control shRNA (shCtr) or ITCH-targeting shRNAs (shiICH-1, shiICH-2), as measured by cell-surface staining and FACS analysis (PD-L2 is not detectable in H358 cells). MFI, mean fluorescence intensity. Mean±SEMs (n=3). P value, Student's t test, ***p<0.001. (9B) Cell-surface levels of PD-L2 in M238 R1 cells stably expressing empty vector (Vec) or over-expression (OE) ITCH, as measured by cell-surface staining and FACS analysis. Mean±SEMs (n=3). P value, Student's t test, ***p<0.001. (9C) Cell-surface levels of PD-L1 in M238R1, H358, and MDA-MB-231 stably expressing control shRNA (shCtr) or ITCH-targeting shRNAs (shiICH-1, shiICH-2) and M238R1 stably expressing empty vector (Vec) or OE ITCH, as measured by immunofluorescence staining. Scale bar=20 μm. (9D) PD-L1 internalization in M238 R1 cells stably expressing empty vector (Vec) or OE ITCH, as measured by FACS analysis. Mean±SEMs (n=3). P value, Student's t test, ***p<0.001. (9E) Confocal microscopic imaging of M238 R1 cells stably expressing empty vector (Vec) or OE ITCH before (0 min) and 30 minutes after initiating surface PD-L1 internalization. Small white arrows, co-localization of PD-L1 and the lysosome marker, LAMP1. Scale bar=10 μm. (9F) M238 R1 cells stably expressing Vec or OE ITCH were treated with indicated concentrations of chloroquine (CQ) (left) or MG-132 (right) for 16 hours, followed by measurement of cell-surface levels of PD-L1 by cell-surface staining and FACS analysis. Mean±SEMs (n=3). (9G) M238 R1 cells stably expressing Vec or ITCH were treated with indicated concentrations of chloroquine (CQ) for 16 hours, followed by measurement of total PD-L1 level by Western blots (WBs). (9H) Real-time PCR of PD-L1 mRNA levels in M238 R1 or H358 stably expressing control and ITCH-shRNAs. Mean SEMs (n=3). P value, Student's t test, ns or not significant. (9I) HEK 293T cells expressing PD-L2-FLAG, with or without ITCH co-transfection, were pre-treated with or without MG-132 (20 μM) for 4 hours followed by anti-FLAG IP and detection of UB by WBs. (9J) HEK 293T cells expressing PD-L1-FLAG, with or without ITCH co-transfection, were subjected to anti-FLAG immunoprecipitation and mass spectrometry analysis. Quantification of the indicated ubiquitination sites on PD-L1 are shown (log 2). P value, Student's t test, ns, not significant. *p<0.05, **p<0.01. (9K) Tandem mass spectra of the ubiquitinated PD-L1-K46 (left) or PD-L1-K162 (right) peptides. (9L) As in 9J, except quantification of K63 ubiquitination on ubiquitin. P value, Student's t test, *p<0.05. (9M) Tandem mass spectrum of the ubiquitinated ubiquitin-K63 peptide. (9N) Jurkat cell-surface PD-1 expression measured by FACS analysis after indicated treatments or co-culture with M238R1-NYESO/A2 cells.

FIG. 10 illustrates a gating strategy for FACS analysis. An example shown following tumor dissociation, with percentages at each step of gating the parental populations.

FIGS. 11A-11B shows the impact of Itch knockdown on in vitro growth of murine melanoma cell lines. (11A) Growth curves of cultured YUMM1.7ER and NILER1-4 cell lines stably expressing control or ITCH-shRNAs (left and middle). Mean±SEMs (n=3). WBs of YUMMER1.7 cell lines stably expressing control or ITCH-shRNAs (right). (11B) Clonogenic growth (10 days) of YUMM1.7ER and NILER1-4 cell lines stably expressing control and ITCH-shRNAs off or on trametinib (Tram) treatment at indicated concentrations.

FIGS. 12A-12G demonstrate the effects of ITCH or PD-L1 expression on immune infiltration or immune cell gene expression. (12A) Tumor growth curves of shCONTROL (shCtr) and ITCH-knockdown (shiICH-3) YUMM1.7ER melanoma under no treatment or on trametinib (Tram, 1 mg/kg/d) treatment in C57BL/6 mice. shCONTROL and ITCH-knockdown tumors (n=3) after 14 days of trametinib treatment were dissociated into single cells followed by CyTOF and scRNA-seq analysis. Mean±SEMs (n=8 for NT groups and n=10 for Tram groups). (12B) Fraction of CD4⁺ or CD8⁺ cells in total live cells from shCONTROL and ITCH-knockdown YUMM1.7ER tumors. Mean±SEMs (n=3). P value, Student's t test, **p<0.01, ns: not significant. (12C) Heatmap showing scaled mean expression levels of indicated markers in different cell clusters of CD4⁺ T cells (YUMM1.7ER). (12D) Heatmap showing scaled mean expression levels of indicated markers in different cell clusters of CD8⁺ T cells (YUMM1.7ER). (12E) Tumor growth curves of shCONTROL (shCtr) and ITCH-knockdown (shiICH mix) NILER1-4 melanoma under trametinib (Tram, 3 mg/kg/d) treatment in C57BL/6 mice. shCONTROL and ITCH-knockdown tumors (n=4) after 5 days of trametinib treatment were dissociated into single cells followed by CyTOF and scRNA-seq analysis. Mean±SEMs (n=8 for NT groups and n=10 for Tram groups). (12F) Heatmap showing scaled mean expression levels of indicated markers in different cell clusters of CD4⁺ T cells (NILER1-4). (12G) Heatmap showing scaled mean expression levels of indicated markers in different cell clusters of CD8⁺ T cells (NILER1-4).

FIGS. 13A-13J show the impact of NILER 1-4 tumor cell-intrinsic ITCH deficiency on single immune cells and gene expression. (13A) UMAP of intra-tumoral CD45⁺ single cells showing expression levels of indicated cell lineage markers (shCONTROL and ITCH-knockdown NILER1-4 tumors, both on trametinib treatment). (13B) UMAP of intra-tumoral CD45⁺ single cells (in A) with indicated cell types denoted by distinct colors. NK (Natural killer cells), TAM (Tumor associated macrophages), DC (Dendritic cells), TAN (Tumor associated neutrophils). (13C) Fractions of indicated cell types in total CD45⁺ cells from NILER1-4 shCONTROL and ITCH-knockdown tumors, both on trametinib treatment. (13D) Heatmap showing scaled mean expression levels of indicated genes in indicated T cell clusters from shCONTROL and ITCH-knockdown NILER1-4 tumors, both on trametinib treatment. (13E) Heatmap showing scaled mean expression levels of indicated genes in indicated TAM cell clusters from shCONTROL and ITCH-knockdown NILER1-4 tumors, both on trametinib treatment. (13F) UMAP of intratumoral TAMs (n=886) analyzed by scRNA-seq (shCONTROL and ITCH-knockdown YUMM1.7ER tumors, both on trametinib treatment). Different cell clusters denoted by distinct colors. (13G) Heatmap showing expression levels of differentially expressed genes (rows) among different TAM subpopulations (columns) in 13F. Representative genes of each cluster are highlighted. (13H) Fractions of each TAM subpopulation in total CD45⁺ cells from shCONTROL and ITCH-knockdown YUMM1.7ER tumors, both on trametinib treatment. (13I) The ratio of M2-like TAMs to M1-like TAMs in shCONTROL and ITCH-knockdown YUMM1.7ER tumors, both on trametinib treatment. (13J) Heatmap showing scaled mean expression levels of indicated genes in indicated TAM cell clusters from shCONTROL and ITCH-knockdown YUMM1.7ER tumors, both on trametinib treatment.

FIGS. 14A-14F show a CyTOF analysis of CD45⁺ and CD4⁺ populations in YUMM1.7ER control and ITCH over-expression tumors. (14A) Clonogenic growth (7 days) of YUMM1.7ER cell lines stably expressing Vec and over-expressing (OE) ITCH, off or on trametinib (Tram) treatment at indicated concentrations. (14B) Tumor growth curves of Vec and ITCH OE YUMM1.7ER tumors on trametinib (Tram, 0.45 mg/kg/d) treatment in C57BL/6 mice. Vec and ITCH OE tumors (n=4 for each condition) before and after 7 days of trametinib treatment were dissociated into single cells and analyzed by CyTOF. Mean #SEMs (n=11-12). (14C) Heatmap showing scaled mean expression levels of indicated protein markers in different cell clusters of CD45⁺ cells (YUMM1.7ER, NT and Tram). (14D) t-SNE map of intra-tumoral CD4⁺ cells from Vec and ITCH-OE YUMM1.7ER tumors on NT and trametinib treatment, as analyzed by CyTOF. Inferred cell types denoted by distinct colors. (14E) Fractions of indicated cell types in CD4⁺ T cells from Vec and ITCH-OE YUMM1.7ER tumors on NT or trametinib treatment. Mean±SEMs (n=4). P value, Student's t test. *p<0.05. (14F) Heatmap showing scaled mean expression levels of indicated protein markers in different cell clusters of CD4⁺ T cells (YUMM1.7ER, NT and Tram).

FIGS. 15A-15B demonstrate that AK087, an ITCH agonist, improves responses of melanoma to immune checkpoint blockade. Data are derived from two murine models of melanoma, YUMM1.ER, Braf^{V600MUT} melanoma, and NILER1-4, Nras^{Q60MUT} melanoma. (15A) YUMM1.7ER. n=9-10 per group. Means±SEM. Anti-PD-1 200 μg/mouse, anti-CTLA-4 200 μg/mouse three times a week for the first two weeks, then change to twice/week treatment. AK087 10 mg/kg/day (Subcutaneously, daily) administration. *p<0.05, student's t test. (15B) NILER1-4. n=8-10 per group. Means±SEM. Anti-PD-1 200 μg/mouse, anti-CTLA-4 200 g/mouse, aLAG-3 200 μg/mouse three times a week for the first two weeks, then change to twice/week treatment. AK087 15 mg/kg/day (Subcutaneously, daily) administration. *p<0.05, student's t test.

FIG. 16. demonstrates that the ITCH agonist AK087 enhances the interaction between UBCH7 and ITCH in HEK293T cells (left panel) and cancer cell lines M229R5, H358, M238R1 and M238 (right panel). HEK293T cells were transfected with HA-UBCH7 and ITCH-FLAG (alone or together) and treated with or without AK087 (100 μM) for 24 or 36 hours, followed by anti-FLAG immunoprecipitation and WB. Right panel: M229R5, H358, M238R1 and M238 cells were treated with DMSO or AK087 (100 μM) for 2 days followed by proximity ligation assay (PLA) using anti-UBCH7 and anti-ITCH antibodies. Signal dots per cell was quantified by the Duolink ImageTool. For bar graph: n=2-3. Means±SEM. *p<0.05, **p<0.01, student's t test.

### DETAILED DESCRIPTION

The invention provides new methods for enhancing cancer immune surveillance and the efficacy of the treatment of melanoma or non-melanoma cancers such as pancreatic or colorectal cancers, particularly by controlling cancer cell-surface PD-L1/L2 and the E3 ligase ITCH. As described herein, the efficacy of MAPK inhibitor therapy is increased by treatment with an activator of E3 ligase ITCH or a destabilizer of cell-surface PD-L1/L2. As further demonstrated herein, the efficacy of anti-PD-1/L1 therapy is also increased by treatment with an activator of E3 ligase ITCH or a destabilizer of cell-surface PD-L1/L2.

### Definitions

All scientific and technical terms used in this application have meanings commonly used in the art unless otherwise specified. As used in this application, the following words or phrases have the meanings specified.

As used herein, “PD-1 (programmed cell death-1)” refers to a receptor expressed on the surface of activated T cells. “PD-L1 and PD-L2” are PD-1 ligands expressed on the surface of dendritic cells, macrophages, or tumor cells. PD-1 and PD-L1/PD-L2 belong to the family of immune checkpoint proteins that act as co-inhibitory factors that can halt or limit activation or persistence of anti-tumor T cell responses.

As used herein, “anti-PD-1 therapy” means treatment with an anti-PD-1 antibody (nivolumab/BMS-936558/MDX-1106, pembrolizumab/MK-3475, Pidilizumab), and/or an anti-PD-L1 antibody (BMS-986559, MPDL3280A, and MEDI4736).

As used herein, “combinatorial therapy” means MAPK targeted therapy, anti-CTLA-4 immunotherapy in any combination, with or without anti-PD-1 antibody, anti-PD-L1 antibody, and/or anti-LAG-3 antibody treatment.

As used herein, “MAPK/ERK kinase (MEK)” refers to a mitogen-activated protein kinase also known as mitogen-activated protein kinase (MAPK) or extracellular signal-regulated kinase (ERK). MAPK, in cancers such as melanoma, pancreatic ductal adenocarcinoma, and colorectal adenocarcinoma, is commonly hyperactivated by mutations in oncogenes (BRAF, NRAS, KRAS), confers tumor growth and survival, and constitutes a pharmacologically targetable pathway.

MEK, also known as mitogen-activated protein kinase kinase and MAP2K, is a kinase enzyme that phosphorylates mitogen activated protein kinases (MAPKs), ERK, p38 and JNK. Seven MEK subtypes have been identified, all mediate cellular responses to different growth signals.

BRAF (v-raf murine sarcoma viral oncogene homolog B1) is a serine/threonine protein kinase that plays a critical role in the RAS-RAF-MEK-ERK mitogen activated protein kinase (MAPK) cell signaling pathway.

As used herein, “proteolysis-targeting chimeras (PROTACs)” refers to bifunctional small molecules that recruit an E3 ligase to a target protein of interest, promoting its ubiquitination and subsequent degradation.

As used herein, “antibody-based PROTACs (AbTACs)” refers to fully recombinant bispecific antibodies that recruit membrane-bound E3 ligases for the degradation of cell-surface proteins. One example of an AbTAC recruits membrane-bound E3 ligases such as RNF43 for the degradation of cell-surface PD-L1 or PD-L2.

As used herein, “therapy”, “treatment” or “treating” means any administration of a therapeutic agent according to the present disclosure to a subject (e.g. human) having or susceptible to a condition or disease, such as cancer, for the purpose of: preventing or protecting against the disease or condition, that is, causing the clinical symptoms not to develop; inhibiting the disease or condition, that is, arresting or suppressing the development of clinical symptoms; or relieving the disease or condition that is causing the regression of clinical symptoms. In some embodiments, the term “therapy”, “treatment” or “treating” refers to relieving the disease or condition, i.e. which is causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment. The presence of a genetic mutation or the predisposition to having a mutation may not be alterable. However, prophylactic treatment (prevention) as used herein has the potential to avoid/ameliorate the symptoms or clinical consequences of having the disease engendered by such genetic mutation or predisposition. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.” The term “effective amount” refers to that amount of a therapeutic agent that is sufficient to effect treatment when administered to a subject in need of such treatment. The effective amount will vary depending upon the specific activity of the therapeutic agent being used, the severity of the patient's disease state, and the age, physical condition, existence of other disease states, and nutritional status of the patient. Additionally, other medication the patient may be receiving will affect the determination of the effective amount of the therapeutic agent to administer.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, PA, 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

### Methods of the Invention

The invention provides methods for improving cancer therapy. The methods are capable of enhancing cancer immune surveillance and the efficacy of the treatment of melanoma and other cancers with MAPK-activating mutations in genes such as BRAF, NRAS, and KRAS, particularly by controlling tumor cell-surface PD-L1/L2 and the E3 ligase ITCH. In addition, the methods enhance the efficacy of immune checkpoint blockade therapies, e.g., anti-PD1/PDL1/PDL2/CTLA4/LAG3 and their combination therapies. In some embodiments, a method of suppressing tumor cell-surface PD-L1 expression to thereby enhance anti-melanoma therapy in a subject in need thereof comprises administering to the subject an effective amount of a modulator of tumor cell-surface PD-L1/L2. In some embodiments, the modulator of tumor cell-surface PD-L1/L2 is an activator of E3 ligase ITCH and/or a destabilizer of tumor cell-surface PD-L1/L2.

In some embodiments, the activator of ITCH is selected from: Chlorophyllide, 3-chloro-1-(4-methylphenyl)-4-piperidin-1-yl-1H-pyrrole-2,5-dione. N-(3,4-dimethylphenyl)benzenesulfonamide, 4-methylphenyl 7-methylpyrrolo[1,2-c]pyrimidin-3-yl sulfone, 5,6,7-trimethoxy-4-methyl-2H-chromen-2-one, and 6-amino-3-methyl-4-{2-[(1-methylethyl)oxy]phenyl}-2,4-dihydropyrano[2,3-c]pyrazole-5-carbonitrile (See Table of Compounds. See also Rossi, et al., Cell Death and Disease 2014, 5, e1203). In some embodiments, the destabilizer of cell-surface PD-L1/L2 is a recombinant bispecific antibody-based proteolysis-targeting chimera (AbTAC) that recruits membrane-bound E3 ligases. Targeted protein degradation has emerged as a new paradigm to manipulate cellular proteostasis. Proteolysis-targeting chimeras (PROTACs) are bifunctional small molecules that recruit an E3 ligase to a target protein of interest, promoting its ubiquitination and subsequent degradation. Cotton, A. D., et al. have described the development of antibody-based PROTACs (AbTACs), fully recombinant bispecific antibodies that recruit membrane-bound E3 ligases for the degradation of cell-surface proteins (J. Am. Chem. Soc. 2021, 143, 2, 593-598).

In some embodiments, the subject is treated with one or more mitogen-activated protein kinase (MAPK) inhibitors. In some embodiments, the subject is treated with one or more mitogen-activated protein kinase (MAPK) inhibitors, and one or more immune checkpoint protein antibodies, such as anti-PD-1/L1 antibodies, as anti-melanoma therapy. In some embodiments, the MAPK inhibitor(s), or MAPK inhibitor(s) plus anti-PD-1/L1/immune checkpoint antibodies, is administered concomitantly with the administering of the activator of ITCH or destabilizer of cell-surface PD-L1/L2. In some embodiments, the MAPK inhibitor(s), or MAPK inhibitor(s) plus anti-PD-1/L1 antibodies/immune checkpoint antibodies, is administered prior to the administering of the activator of ITCH or destabilizer of cell-surface PD-L1/L2. In some embodiments, the MAPK inhibitor(s) or MAPK inhibitor(s) plus anti-PD-1/L1/immune checkpoint antibodies is administered subsequent to the administering of the activator of ITCH or destabilizer of cell-surface PD-L1/L2. In some embodiments, the MAPK inhibitor is selected from: Vemurafenib, Dabrafenib, Encorafenib, Trametinib, Binimetinib, and Cobimetinib, as well as type II RAF inhibitors or pan-RAF inhibitors, such as BGB-283, BGB-3245, DAY101/TAK-580, KIN-2787, and LXH254.

Also described herein is a method of inhibiting an adaptive immune resistance response to anti-PD-1/L1 therapy or other immune checkpoint therapy in a subject in need thereof. In some embodiments, the method comprises administering to the subject an effective amount of an activator of E3 ligase ITCH or a destabilizer of cell-surface PD-L1/L2. Examples of such activators and destabilizers are described herein. In some embodiments, the immune checkpoint therapy comprises anti-CTLA-4 antibody, anti-PD-1 antibody, anti-PD-L1 antibody, and/or anti-LAG-3 antibody treatment.

### EXAMPLES

The following examples are presented to illustrate the present invention and to assist one of ordinary skill in making and using the same. The examples are not intended in any way to otherwise limit the scope of the invention.

### Example 1: Enhancing PD-L1 Degradation by ITCH During MAPK Inhibitor Therapy Suppresses Acquired Resistance

MAPK inhibitor (MAPKi) therapy in melanoma leads to accumulation of tumor-surface PD-L1/2, which may evade antitumor immunity and accelerate acquired resistance. This Example demonstrates that the E3 ligase ITCH binds, ubiquitinates, and down-regulates tumor-surface PD-L1/L2 in MAPKi-treated human melanoma cells, thereby modulating activation of co-cultured T cells. During MAPKi therapy in vivo, tumor cell-intrinsic ITCH knockdown in murine melanoma induced tumor-surface PD-L1, reduced intratumoral cytolytic CD8⁺ T cells, and accelerated acquired resistance only in immune-proficient mice. Conversely, tumor cell-intrinsic ITCH over-expression reduced MAPKi-elicited PD-L1 accumulation, augmented cytolytic CD8⁺ T-cell infiltration, and suppressed acquired resistance in Braf^{MUT}, Nras^{MUT}, and Nf1^{MUT} murine melanoma and Kras^{MUT} pancreatic cancer models. CD8⁺ T-cell depletion and tumor cell-intrinsic PD-L1 over-expression nullified the ability of ITCH over-expression to suppress MAPKi-resistance, supporting in vivo the ITCH-PD-L1-T-cell regulatory axis demonstrated in human cancer cell lines. Moreover, we identified a small-molecular ITCH activator which suppressed acquired MAPKi-resistance in vivo. Thus, MAPKi-elicited tumor-surface PD-L1 accelerates acquired-resistance, and degrading PD-L1 by activating ITCH may be a combinatorial approach to promote antitumor T-cell immunity and durable responses.

MAPKi induces tumor cell-surface PD-L1 accumulation, which promotes immune evasion and therapy resistance. ITCH degrades PD-L1, optimizing anti-tumor T-cell immunity. This Example shows that degrading tumor cell-surface PD-L1 and/or activating tumor-intrinsic ITCH provide strategies to overcome MAPKi resistance.

Disrupting PD-L1 interaction with PD-1 rejuvenates antitumor immunity and elicits clinical antitumor responses across a wide-range of cancer histologies (1). Cancer cells can express robust surface levels of PD-L1 to tolerize tumor-specific T cells, but regulation of PD-L1 protein levels on the tumor cell surface is poorly understood. De-differentiated or quasi-mesenchymal tumor cells up-regulate PD-L1/L2 (or vice versa) and induce an immune-suppressive microenvironment, including expansion of M2-like macrophages and regulatory T cells as well as depletion of CD8⁺ T cells (2). Targeted therapy, including MAPKi therapy in melanoma, leads to de-differentiation, PD-L1/2 upregulation, and resistance (3), and both MAPKi treatment and mesenchymal signatures are associated with innate anti-PD-1 resistance (4,5).

Tumor cell expression of PD-L1 is induced by transcriptional mechanisms, e.g., in response to inflammatory cytokines such as interferon-g (IFNg) or tumor necrosis factor-a (TNFa) or by natural selection via immune-editing, e.g., gene amplification of PD-L1 (6) and structural variation of the 3′ untranslated region (7). More recent studies have implicated post-genomic and -transcriptomic mechanisms, including how tumor cell-intrinsic control of PD-L1 protein stability regulates antitumor immunity (8-11). Our previous work showed that clinical melanoma induces PD-L1/L2 protein levels early on MAPKi therapy (3), suggesting a mechanism of immune evasion that reduces the efficacy of targeted therapy and that may be ameliorated by combining anti-PD-1/L1 therapy. Indeed, in murine models of Braf^{MUT} and Nras^{MUT} melanoma, introduction of high mutational burden (and presumably neoantigens) extends the durability of MAPKi responses in a fashion dependent on CD8⁺ T cells (12,13), and swiftly sequencing anti-PD-1/L1 ahead of combination with MAPKi therapy strongly suppressed acquired MAPKi resistance (13).

To understand how dynamic PD-L1/L2 protein levels are regulated via protein-protein interactions in MAPKi-resistant melanoma cells, we took two complementary affinity-purification mass-spectrometry approaches. From these data, we prioritized a common hit, the E3 ligase ITCH, for validation as a PD-L1 interaction partner, determined its regulation of PD-L1 at the tumor cell-surface, and characterized PD-L1 poly-ubiquitination by ITCH, thereby targeting PD-L1 to the lysosomal degradation pathway. We also investigated the impact of PD-L1-ITCH interaction on T-cell activation in co-cultures and on in vivo MAPKI resistance using multiple melanoma models in syngeneic and immune-compromised hosts. In additional mechanistic studies, we dissected how perturbing tumor-intrinsic ITCH expression during MAPKi treatment impacts the tumor immune microenvironment and investigated the critical contribution of CD8⁺ T cells to the tumor-extrinsic function of ITCH expressed by melanoma cells. Lastly, we provided proof-of-concept data in support of the feasibility of identifying an ITCH activator that can suppress acquired MAPKi-resistance in vivo.

### Methods

### Cell Lines

All human and mouse cancer cell lines were routinely tested for mycoplasma and profiled and identified by RNA-seq and the GenePrint 10 system (Promega) at periodic intervals during the course of this study. The human cell lines (HEK 293T, M229 R5, M238 R1, H358, and MDA-MB-231) and mouse cell lines (mSK-Mel254, KPC) were maintained in high glucose DMEM with 10% heat-inactivated FBS (Omega Scientific) and 2 mM glutamine. 1 μM of PLX4032 was added in the culture medium of M229 R5 and M238 R1. YUMM1.7ER cell line was maintained in DMEM/F12 with 10% heat-inactivated FBS and 2 mM glutamine. NILER1-4 cell line was cultured in high glucose DMEM with 20% heat-inactivated FBS and 2 mM glutamine. Jurkat T cells were cultured in RPMI1640 medium with 10% heat-inactivated FBS, 10 mM HEPES, 1 mM sodium pyruvate, 2 mM glutamine and 50 μM beta-mercaptoethanol. hPBMCs were maintained in RPMI1640 medium with 10% heat-inactivated FBS, 10 mM HEPES, 1 mM sodium pyruvate, and 2 mM glutamine. All cell lines were maintained in humidified, 5% CO2 incubator.

### Constructs and Engineered Cell Lines

For overexpression constructs, cDNAs of human and mouse PD-L1, PD-L2, and/or ITCH were subcloned into the lentivirus vector cPPT-puro with or without C-terminal tag (FLAG or HA) as indicated. APEX2 coding sequence was fused to the C-terminus of human PD-L1 with the linker peptide GGGGSGGGGS (SEQ ID NO: 1) and subcloned into the lentivirus vector pLV-puro. shRNAs were constructed into the lentivirus vector pLKO.1-puro. Stable lines were selected by adding 10 mg/mL puromycin in culture medium 48 hours after lentiviral infection. shRNA targeting sequences are as follows:

Human ITCH sh1:
(SEQ ID NO: 2)
CGAAGACGTTTGTGGGTGATT
Human ITCH sh2:
(SEQ ID NO: 3)
GCCTATGTTCGGGACTTCAAA
Mouse ITCH sh2:
(SEQ ID NO: 4)
GCAGCAGTTTAACCAGAGATT
Mouse ITCH sh3:
(SEQ ID NO: 5)
AATCCAGACCACCTGAAATAC
Control shRNA:
(SEQ ID NO: 6)
CCTAAGGTTAAGTCGCCCTCG

### IP-MS Sample Tryptic Digestion

PD-L2 with C-terminal FLAG-tag, or vector Flag IP samples (for PD-L2 interactome analysis), or PD-L1 C-terminal FLAG-tag co-transfected with/without ITCH (for PD-L1 ubiquitination analysis) were subjected to immunoprecipitation. After elution in buffer (0.1 M glycine-HCl, pH 3.0), eluates were reduced and alkylated by sequentially incubating with 5 mM TCEP and 10 mM iodoacetamide (chloroacetamide, for ubiquitination analysis) for 30 minutes at room temperature in the dark. The samples were then incubated overnight at 37″ C with Lys-C and trypsin protease at ratios of 1:100 each. Peptide digests were desalted using Pierce C18 tips (100 ml bed volume, cat. 87784), dried by vacuum centrifugation, and reconstituted in 5% formic acid.

### APEX2-Based Proximity Labeling

PD-L1-APEX2-expressing cells were cultured as previously described. 500 mM biotin-phenol was added to the media and incubated at 37° C. for 30 minutes. The peroxidase reaction was activated by adding H₂O₂ (no H₂O₂ was added to the negative control) to 1 mM and incubating at room temperature for 1 minute. The reaction was quenched by washing cells three times with a quencher-containing PBS (10 mM sodium azide, 5 mM Trolox, 10 mM sodium ascorbate). Cells were harvested by trypsinization and then flash-frozen in liquid nitrogen.

### Streptavidin Pull-Down

Cells were lysed in RIPA buffer (50 mM Tris-HCl PH7.5, 150 mM NaCl, 0.1% SDS, 0.5% sodium deoxycholate, 1% TritonX-100) supplemented with protease inhibitor cocktail (Roche) and Benzonase (1 ml of 250 U/μl) and incubated at 37° C. for 20 minutes. Lysates were clarified by centrifugation, quantitated using the Pierce 660 nm protein assay, and 1 mg of protein was incubated with 300 ml of high-capacity streptavidin beads (Thermo Fisher) for each sample at room temperature for one hour. Streptavidin beads were then washed three times with RIPA buffer, once with 1 μM KCl, once with 2 μM Urea in 25 mM Tris-HCl PH=8.0, and 3 more times with RIPA buffer. Bound proteins were then reduced, alkylated, and digested on beads with Lys-C and trypsin. The supernatant from the on-bead digestion was then transferred to another tube, bound to SP3/CMMB beads by the addition of acetonitrile to a concentration of 95%, and eluted in 0.1% formic acid.

### LC-MS Data Acquisition

Samples were loaded onto a 75 μm×25 cm homemade C18 column connected to a nano-flow Dionex Ultimate 3000 UHPLC system and fractionated online using a 140-minute gradient of increasing acetonitrile (ACN) delivered at a 200 nl/min flow rate. An Orbitrap Fusion Lumos Tri-brid mass spectrometer was used for data acquisition using data-dependent acquisition (DDA) mode. Full MS scans were acquired at 120K resolution with the AGC target set to 2e5 and a maximum injection time set to 100 ms. MS/MS scans were collected at 15K resolution after isolating precursors with an isolation window of 1.6 m/z and HCD-based fragmentation using 35% collision energy. For data dependent acquisition, a 3-second cycle time was used to acquire MS/MS spectra corresponding to peptide targets from the preceding full MS scan. Dynamic exclusion was set to 25 seconds.

### Database Search

MS/MS database searching was performed using MaxQuant (1.6.17.0) against the human reference proteome from EMBL (UP000005640_9606 HUMAN Homo sapiens, 20600 entries, released in 2020_04). The search included carbamidomethylation on cysteine as a fixed modification and methionine oxidation and N-terminal acetylation as variable modifications; identification of ubiquitination sites was searched with di-Gly modification as a variable modification in addition to the aforementioned ones. The digestion mode was set to trypsin and allowed a maximum of 2 missed cleavages. The precursor mass tolerances were to 20 and 4.5 ppm for the first and second searches, respectively, while a 20-ppm mass tolerance was used for fragment ions. Datasets were filtered at 1% FDR at both the PSM and protein-level. Peptide quantitation was performed using MaxQuant's LFQ mode. Visualization of the ubiquitinated peptide-spectrum match was conducted by parsing MaxQuant search results through PDV (32).

### Statistical Analysis of Proteomics Data

PD-L2-Flag IP dataset was searched with MaxQuant, and the resulting MS/MS spectral count information was used with SAINTexpress⁴ (v3.6.3) to generate a protein interaction confidence score. MSStats (3.10) was used to analyze the MaxQuant LFQ data in the PD-L1-APEX2 proximity labeling experiment to statistically assess protein enrichment. Equalized medians were used for normalization, and the Tukey median polish method was used for protein summarization. P-values for t-tests were corrected for multiple hypothesis testing using the Benjamini-Hochberg adjustment.

### Western Blot and Immunoprecipitation

Cells were lysed in IP lysis buffer (Thermo Fisher Scientific) with protease and phosphatase inhibitor cocktail (Thermo Fisher Scientific) for immunoprecipitation (IP) and Western blotting. Dynabeads pre-incubated with antibody or anti-FLAG M2 beads (Thermo Fisher Scientific) were used to immunoprecipitate proteins of interest based on the manufacturer's protocol. Antibodies used in IP and Western blot are as follows: TUBULIN (Sigma Aldrich, T9026), ITCH (BD, 611198), HA (CST, C29F4), FLAG (CST, 2368S), PD-L1 (CST, E1L3N), GAPDH (CST, D16H11), UBIQUITIN (CST, 3933S; CST, PD41).

### Cell Surface PD-L1 and PD-L2 Detection

For cultured cell lines, cells were detached from culture dish using trypsin-free detachment solution Accutase (BioLegend, 423201) to preserve cell surface proteins. 5×10⁵ cells were stained for either APC-anti-PD-L1 (1 mL/sample, BioLegend, 329708), PE-anti-PD-L2 (1 mL/sample, BioLegend, 329606), or both followed by flow cytometry analysis to quantify the mean fluorescent intensity (MFI). For subcutaneous tumor models, tumors were dissociated to single-cell suspensions using a tumor dissociation kit and gentleMACS™ Octo Dissociator (Miltenyi Biotec). 1×10⁶ cells were incubated with 20% of FBS in PBS with 25 mg/ml of anti-mouse CD16/CD32 (clone 2.4G2) antibody at 4° C. for 10 min to minimize non-specific binding prior to surface staining with BV510-anti-CD45 (1 mg/mL, BioLegend, 103138), BV421-anti-CD90 (2 mg/mL, BioLegend, 328122), APC-anti-PD-L1 (2 mg/mL, BioLegend, 124312), PE-anti-PD-L2 (2 mg/mL, BioLegend, 107206), and PerCP-anti-TER 119 (2 mg/mL, BioLegend, 116226) at room temperature for 20 minutes, followed by 7AAD (10 mL in 500 mL PBS per sample, Beckman Coulter, A07704) staining for 5 minutes on ice. Live tumor cells were gated as BV510⁻/BV421⁻/PerCP⁻ population (CD45/CD90), and MFIs of APC (PD-L1) and PE (PD-L2) were measured by flow cytometry analysis. Average PD-L1 expression of the CD45⁺ population was used as an internal control to normalize measurements of tumor surface PD-L1 levels on different days.

### Ubiquitination Assay

PD-L1 ubiquitination assay was performed following the protocol of Signal-Seeker™ Ubiquitination Detection Kit (Cytoskeleton, BK161). In bnef, cells were treated with 20 μM MG-132 for 4 hours followed by cell lysis with protease and de-ubiquitination inhibitors. Cell lysates were purified by passing through a filter, and immunoprecipitation was performed using Dynabeads (pre-incubated with anti-PD-L1) or anti-FLAG M2 beads Ubiquitination on the target protein was detect by Western Blot using anti-UBIQUITIN antibody (CST, 3933S; CST, PD41).

### PD-L1 Internalization Assay

Assay was performed as described previously (8). In brief, cell surface PD-L1 was labelled with unconjugated anti-PD-L1 (BioLegend, 29E.2A3) for 1 h on ice and washed twice to remove unbound antibody. Cells were resuspended in culture medium on ice, and a baseline sample removed and kept on ice. Cells were incubated at 37° C. in a water bath and removed at the indicated times followed by immediately dilution in ice-cold PBS to stop further endocytosis. Cells were washed twice, and the remaining cell surface-bound anti-PD-L1 antibodies were stained with Alexa Fluor-488-conjugated anti-mouse secondary antibody (Invitrogen, A11001) for 30 min on ice. Finally, samples were washed twice and analyzed by flow cytometry.

### Microscopy and Fluorescent Imaging

For imaging by confocal microscopy, cells were plated in 8-chamber cell culture slide at 50-80% confluence. Anti-PD-L1 (BioLegend, 29E.2A3) was added at 1:100 dilution to label cell-surface PD-L1 at room temperature for 20 mins. Cells were washed 3 time with PBS, and the sample wells for 0 min internalization were fixed by methanol for 20 mins at −20° C. The sample wells for PD-L1 internalization were transferred into a 37° C. cell culture incubator with full medium for 30 mins, followed by PBS wash for 3 times and fixation by methanol for 20 mins at −20° C. Then, standard immunofluorescence staining protocol was applied with Alexa Fluor-647-conjugated anti-LAMP1 (1:100, 4° C. overnight; CST, 73589) and Alexa Fluor-488-conjugated anti-mouse secondary antibody (1:200, room temperature, 1 hour; Invitrogen, A11001) for the samples before and after PD-L1 internalization. The slides were mounted in antifade solution with DAPI (Invitrogen, P36935). Confocal images were taken by ZEISS LSM 880 63X (oil) objective at the UCLA CHS confocal microscopy core.

For general immunofluorescence imaging of PD-L1 expression in cell lines, cells were plated in a 8-chamber cell culture slide at 50-80% confluence. Standard immunofluorescence staining protocol was performed with methanol fixation (−20° C., 20 mins) and Alexa Fluor-555-conjugated anti-PD-L1 (1:50, 4° C. overnight; CST, 40216). The slides were mounted in antifade solution with DAPI (Invitrogen, P36935), and images were taken by a Zeiss microscope (AXIO Imager A1, 40× objective) mounted with a charge-coupled device camera (Retiga EXi QImaging).

### Real-Time PCR

One million cells were subjected to total RNA extraction, reverse transcription, and cDNA quantification using SYBR Green method by the MyiQ Real-Time PCR Detection System (Bio-Rad). Relative expression of PD-L1 was calculated using the delta-Ct method and normalized to TUBULIN levels. The sequence of PCR primers used are as follows:

PD-L1-F:
(SEQ ID NO: 7)
TGCCGACTACAAGCGAATTACTG
PD-L1-R:
(SEQ ID NO: 8)
CTGCTTGTCCAGATGACTTCGG
TUBULIN-F:
(SEQ ID NO: 9)
GCACGATGGATTCGGTTAGGTC
TUBULIN-R:
(SEQ ID NO: 10)
TCGGCTCCCTCTGTGTAGTGG

### Co-Culture Assay

Target cell lines expressing NYESO-HLA-A2 were plated on 24-well plates at a concentration of 1×10⁵ per well. 12 to 16 hours later, media were changed, and Jurkat T cells expressing TCR (1G4) or human PBMCs (ATCC, PCS-800-011) were added to the culture wells at a concentration of 1×10⁶/mL (400 μL) for 24 hours. Anti-CD3 (Invitrogen, 16-0037-81) and anti-CD8 (Invitrogen, 16-0289-81) were added into the culture media at a final concentration of 1 μM (when using hPBMCs). Anti-PD-1 (BioLegend, 329925) and anti-PD-L1 (BioLegend, 329715), when applicable, were added in the culture media at a final concentration of 1 μM. Media were harvested after co-culture and diluted from 1/10 to 1/50 and subjected to ELISA assay to detect IL-2 production (BioLegend, 431804).

### Subcutaneous Syngeneic Tumor Models

Animal research in this study has been approved by the local Animal Research Committee. C57BL/6 and NSG mice were obtained from the Radiation Oncology breeding colony at UCLA (Los Angeles, CA). Female mice were used at 6-8 weeks of age. All animal experiments were conducted according to the guidelines approved by the UCLA Animal Research Committee. For subcutaneous tumor models, C57BL/6 or NSG (YUMM1.7ER, NILER1-4, mSK-Mel254, KPC) mice were injected on both flanks with one million cells per injection. Tumors were measured with a calliper every 2 or 3 days, and tumor volumes were calculated using the formula (length×width²)/2. Once tumors reached a size of 100-150 mm³, mice were assigned randomly into experimental groups. Special mouce diets (for C57BL/6 or NSG) were generated by incorporating trametinib at 0.45, 1, or 3 mg/kg/d or PLX4032 50 mg/kg/d plus trametinib 0.3 mg/kg/d (for the BRAFi+MEKi combination) to facilitate daily drug dosing and to reduce animal stress (TestDiet, Richmond, IN, USA). Anti-PD-L1 (200 mg/mouse) (BioXcell 10F.9G2, Lebanon, NH, USA) was intraperitoneally administered twice per week. Anti-CD8 (200 mg/mouse) (BioXcell, YTS 169.4) was intraperitoneally administered twice per week starting from one day before trametinib treatment. RP-832c was subcutaneously administrated daily (10 mg/kg) (Riptide Bioscience) from day 0 to day 7 simultaneous with starting trametinib treatment. AK087 was dissolved in vehicle (4% Tween80, 8% DMSO in ddw) and subcutaneously administered near the tumor daily (10 mg/kg) starting with trametinib treatment but only from day 1 to day 17. Tumors were excised from mice, minced, and digested to single-cell suspensions using a tumor dissociation kit and gentleMACS™ Octo Dissociator (Miltenyi Biotec), sorted (by 7-AAD; ThermoFisher Scientific), and prepared for scRNA-seq and/or CyTOF analysis.

### Mass Cytometry of Murine Tissues

2×10⁶ or fewer cells were incubated with 20% of FBS in PBS with 25 mg/ml of anti-mouse CD16/CD32 (clone 2.4G2) antibody at 4° C. for 10 min to minimize non-specific binding prior to surface staining with an antibody cocktail at 4° C. for 30 min in a 50 ml volume. Cells were incubated with 2.5 mM 194 Pt monoisotopic cisplatin (Fluidigm) at 4° C. for 1 min. Cells were then washed twice with FACS buffer and barcoded using palladium metal barcoding reagents according to manufacturer's protocol (Fluidigm). Subsequently, fixation and permeabilization were performed using the FOXP3 fix and permeabilization kit according to the manufacturer's protocol (eBioscience). Cells were then stained with an intracellular stain antibody cocktail (FOXP3, Ki67, Granzyme B, T-bet, INOS, EOMES) for 30 min at room temperature. Subsequently, cells were washed twice with FOXP3 permeabilization buffer, twice with FACS buffer, and incubated overnight in 1.6% PFA in PBS with 100 nM iridium nucleic acid intercalator (Fluidigm). Finally, cells were washed twice with PBS with 0.5% BSA, filtered, and washed twice with water with 0.1% BSA prior to analysis. Samples were analyzed using a Helios mass cytometer based on the Helios 6.5.358 acquisition software (Fluidigm).

### CyTOF Data Analysis

All the samples were pre-processed by CATALYST, including normalization, debarcoding and compensation. The normalized fcs files were then uploaded into Cytobank (33), and data were gated to exclude beads and to only include live, single cells. CD8⁺ and CD4⁺ T cells were gated from the CD45⁺ CD3⁺ populations, and respectively data were downloaded separately into individual files for each sample. We applied Cytofkit (34) to perform the t-Distribution Stochastic Neighbor Embedding (t-SNE) analysis separately on the manually gated CD4⁺ and CD8⁺ populations from tumor samples. We selected 5,000 events/sample (all events if less than 5,000) to ensure equal representation of cells across samples. For CD4⁺ T cells, 12 markers, including CD44, CD62L, CD25, CD69, CD366, FOXP3, PD-1, CTLA-4, ICOS, EOMES, T-bet and Ki67, were used to cluster the cell populations. For CD8⁺ T cells, CD44, CD62L, CD25, CD69, CD366, Granzyme B, PD-1, CTLA-4, ICOS, EOMES, T-bet and Ki67 were used. We chose 1,000 iterations, perplexity of 30, and theta of 0.5 as the standard t-SNE parameters. Mean intensity values of markers in each cluster were calculated and visualized via heatmaps. Cells were assigned to different populations on the basis of the local gradient expression of known markers. Numbers of cells and percentages of different immune cell subsets were calculated for each sample.

### Single Cell 5′ Gene Expression and V (D) J Sequencing

Three or four different tumors were dissociated to single-cell suspensions using a tumor dissociation kit (Miltenyi Biotech, 130-095-929) and gentleMACS™ Octo Dissociator (Miltenyi Biotec, 130-095-937). Equal number of cells per tumor were pooled together (2×10⁶ total per sample). Cells were incubated with 20% FBS in PBS with 25 mg/mL of anti-mouse CD16/CD32 antibody (Invitrogen, 14-0161-86) at 4° C. for 10 min to minimize background antibody binding. Then, cells were stained with BV510-anti-CD45 (1 mg/mL, BioLegend, 103138) and PerCP-anti-TER119 (2 mg/mL, BioLegend, 116226) at room temperature for 20 minutes, followed by 7AAD (10 mL in 500 mL PBS per sample, Beckman Coulter, A07704) staining for 5 minutes on ice. Cells after staining were sorted by BD FACSAria II sorting system to harvest the BV510 (CD45) positive and PerCP (TER119, 7AAD) negative populations. Cells recovered were subjected to the 10× Genomics standard protocol for coupled scRNA-seq and scTCR-seq library preparation using Chromium Next GEM Single Cell 5′ Library and Gel Bead Kit v1.1 (10× Genomics, 1000167) and V (D) J Enrichment Kit for Mouse T Cells (10× Genomics, 1000071). Libraries were sequenced by NovaSeq 6000 S2 flow cell with 2×50 reads targeting a minimum of 20,000 read pairs per cell for scRNA-seq library and 5,000 read pairs per cell for scTCR-seq library.

Analysis of scRNA-Seq Data

Alignment to GRCm38 reference genome, barcode, and unique molecular identifier (UMI) counting were performed using Cell Ranger (10× Genomics, v2.1.0). Seurat package (35) was used for downstream analysis. Cells with fewer than 200 genes detected or greater than 20% mitochondrial RNA content were excluded from further analysis. Raw UMI counts were normalized to UMI count per million total counts and log-transformed. Variable genes were detected based on average expression and dispersion for each dataset independently. We then use the CellCycleScoring function to calculate scores of S and G2/M cell cycle phases for each cell. Single cells from different conditions were integrated into a single assay based on variable genes identified from each sample. We then use the ScaleData function to calculate scaled z-scores of each variable gene in the integrated assay and regress out the effect of number of genes per cell, mitochondrial RNA content, and cell cycle scores (S phase score and G2/M phase score). This scaled data set was then used for principal component analysis (PCA) of cells. Clusters and UMAP projections were generated based on the top 30 PCA dimensions. Clusters were annotated based on expression of known marker genes, including Cd14 (myeloid), Igtam, Csf1r (monocyte/macrophage), Fit3 (dendritic cell), S100a8, S100a9 (neutrophil), Ncr1 (NK cell), Cd19, Cd79a (B cell), Cd3d, Cd3e, Cd3g (T cell). Cell clusters co-expressing markers of multiple cell types were defined as doublets and excluded from further analysis. We next isolated the monocyte/macrophage and T cell populations identified from the broad clustering analysis and performed re-clustering analysis on them separately. Cells were re-clustered as described above and functional subpopulations were inferred and annotated by identifying differentially expressed marker genes with log-fold changes higher than 0.4 using MAST in the FindAllMarkers function. The M2/M1-like TAM ratios in the macrophage population were calculated as the ratio of proportions between the inferred anti-vs. pro-inflammatory subpopulations.

Analysis of scTCR-Seq Data

Alignment to the GRCm38 reference genome and TCR contig annotation were performed by Cell Ranger vdj pipeline (10× Genomics, v2.1.0). For the TCR clonotype analysis, only cells assigned with both productive TRA and TRB sequences were kept for further analysis. If one cell had two or more TRA-TRB pairs identified, the pair with higher UMIs was considered as the dominant TRA-TRB pair in the corresponding cell and used in the analysis. We defined each unique TRA-TRB pair as a clonotype. The clonal status of TCR clones were characterized as non-clonal (n=1) and clonal (n≥2) based on their cell numbers. The TCR clonotype of each cell was further linked to inferred functional subsets based on the barcode information. We used the STARTRAC package (36) to estimate the expansion and transition index of distinct T cell subsets.

### Analysis of Patient-Derived, Public Datasets

Using web server TIMER2.0 (timer.comp-genomics.org/) (37), a data set of 471 cutaneous melanoma patients or 533 renal clear cell carcinoma patients from the TCGA database were split into top n % patients with high intratumoral ITCH expression and bottom n % patients with low intratumoral ITCH expression to generate patient survival curve (n % indicated in figure legends). Difference between groups was calculated by both Cox Proportional Hazard Model and log rank test. Significant difference was defined as p<0.05. Analysis of correlations between intratumoral PD-L1 expression and TREG infiltration or between ITCH RNA levels and CD8⁺ T-cell infiltration levels in tumor-derived transcriptome data was also performed by TIMER2.0 with algorithms CIBERSORT-ABS (38), QUANTISEQ (39), XCELL (40), TIMER (40) or EPIC (42). A data set of 471 cutaneous melanoma patient samples was analyzed with tumor purity adjustments. Significant difference for Spearman's correlation analysis was defined as p<0.05.

For pan-cancer correlation analysis between ITCH mRNA levels and PD-L1 protein levels, level-3, normalized mRNA expression data was downloaded from the TCGA data portal (https://portal.gdc.cancer.gov/) and level-4 normalized PD-L1 protein expression data was downloaded from The Cancer Proteome Atlas (TCPA) database (tcpaportal.org/tcpa/download.html). Two datasets were merged by matching the sample names, and the Spearman's correlation score was calculated (n=7.194). Tumor samples were ranked by ITCH mRNA expression levels and split into the top (ITCH high) and bottom (ITCH low) 20% (or 50%, as indicated in the figure legends) groups to compare PD-L1 protein levels.

Raw sequencing files of scRNA-seq and scTCR-seq are available at the Gene Expression Omnibus (GEO). The raw files of mass spectrometry data and mass cytometry data are uploaded to FlowRepository (flowrepository.org/). Raw files of mass spectrometry data are available at the Proteomics Identifications Database (ebi.ac.uk/pride/).

### Results

PD-L1/L2—Highly Expressed by MAPKi-Adapted Melanoma-Interact with ITCH

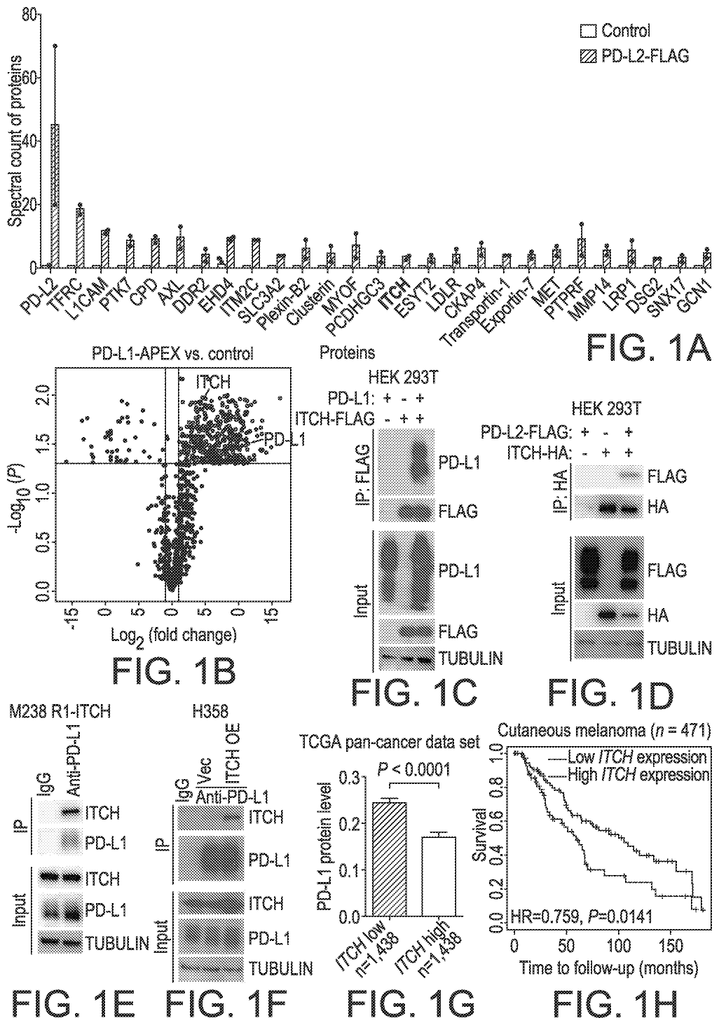

First, using a human melanoma cell line (M229 R5) (14) with acquired resistance to a BRAF^{V600MUT} inhibitor (vemurafenib), we engineered via lentiviral stable transduction PD-L2-FLAG expression and performed anti-FLAG immunoprecipitation followed by mass spectrometry. Among the top hits is an E3 ligase, ITCH (FIG. 1A), which is not known to regulate antitumor immunity or therapy resistance. Second, since artifactual protein interactions can occur or physiologic interactions can be lost after cell membrane solubilization, we surveyed the in situ neighborhood protein interactome of PD-L1 in live melanoma cells. We engineered M229 R5, via lentiviral stable transduction, to express the bacterial biotinylation enzyme (APEX2) fused to the C-terminus of PD-L1. Mass spectrometry quantification of biotin-labeled proteins in cell treated (vs. not treated) with the APEX2 activator H₂O₂ identified ITCH as a PD-L1 proximity interaction partner (FIG. 1B).

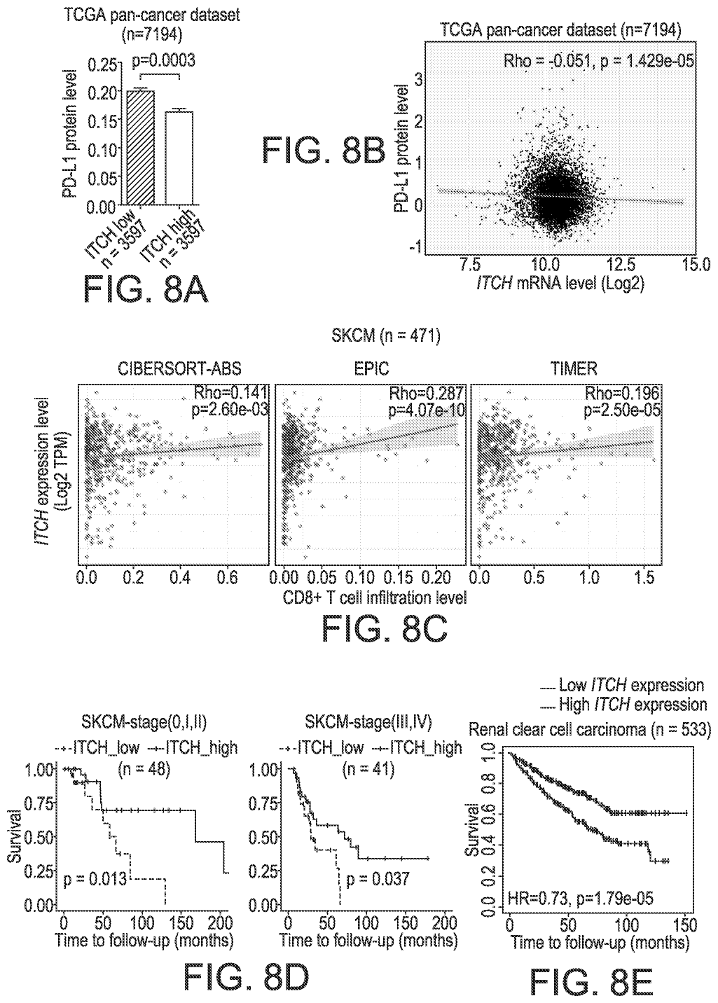

To corroborate the hypothesis that PD-L1 or PD-L2 physically interact with ITCH, we used HEK 293T cells to over-express PD-L1, ITCH-FLAG, or PD-L1+ITCH-FLAG (FIG. 1C) or PD-L2-FLAG, ITCH-HA, or PD-L2-FLAG+ITCH-HA (FIG. 1D). After immunoprecipitation against the FLAG epitope, we detected PD-L1 only when PD-L1 and ITCH-FLAG were co-transfected (FIG. 1C). After immunoprecipitation against the HA epitope, we detected PD-L2-FLAG only when PD-L2-FLAG and ITCH-HA were co-transfected (FIG. 1D). To corroborate the interaction of PD-L1 with ITCH in additional cancer cell lines, we used stable lentiviral transduction to express ITCH in another human melanoma cell line with acquired resistance to a BRAFV600^{MUT} inhibitor (M238 R1) (14) and a human non-small cell lung carcinoma cell line, H358. We chose H358 based on its relatively high basal level of PD-L1 protein expression, despite its lack of detectable PD-L2 protein expression by Western blotting or flow cytometry. Indeed, when we immunoprecipitated against endogenous PD-L1, we could detect stably transduced ITCH (FIGS. 1E and 1F) or endogenous ITCH (FIG. 1F). Given the above evidence of physical interaction between ITCH and PD-L1, we evaluated the pan-cancer relationship between bulk tumor ITCH RNA expression levels and PD-L1 protein levels from The Cancer Proteome Atlas data set (15). Consistent with the E3 ligase ITCH being a potential negative regulator of PD-L1, we observed that higher expression of ITCH RNA is associated with lower PD-L1 protein levels (FIG. 1G; FIG. 8A), and their levels are anti-correlated (FIG. 8B). Consistent with the T-cell-suppressive role of PD-L1, we detected positive correlations between ITCH expression and CD8⁺ T-cell infiltration levels by analyzing the The Cancer Genome Atlas (TCGA) skin melanoma RNA-seq data (FIG. 8C). Accordingly, in TCGA skin melanoma (FIG. 1H; FIG. 8D) and renal clear cell carcinoma (FIG. 8E), tumors with higher ITCH RNA levels are associated with improved survival, suggesting that higher ITCH protein levels, via interaction with and degradation of PD-L1/L2, may enhance CD8⁺ T-cell infiltration and tumor immune surveillance.

### ITCH Ubiquitinates and Down-Regulates Tumor-Surface PD-L1

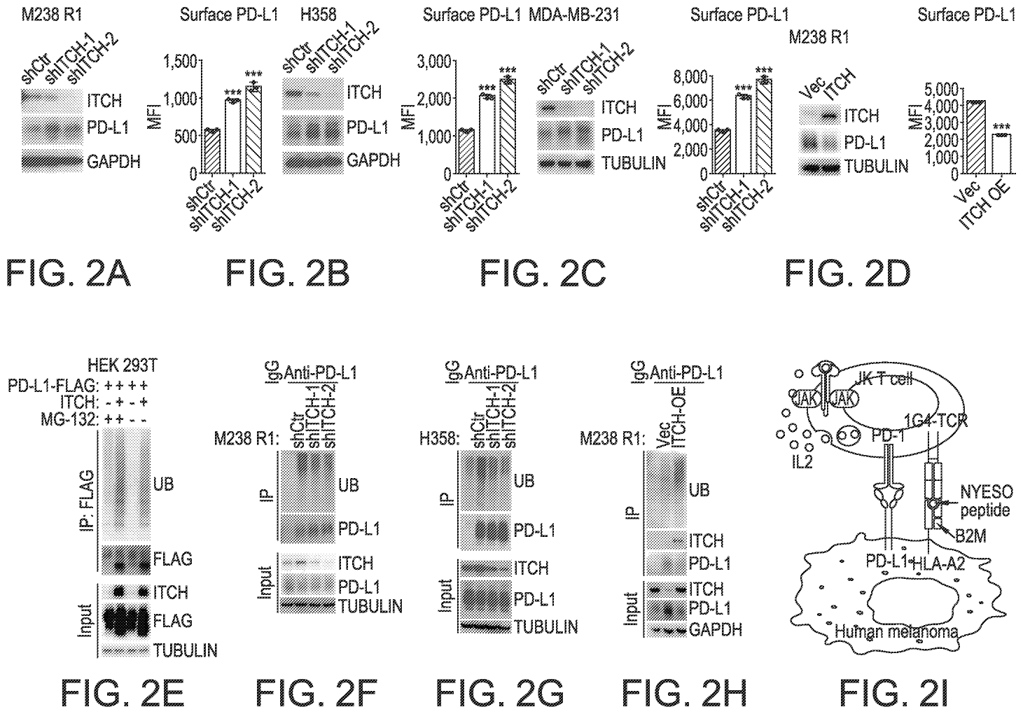

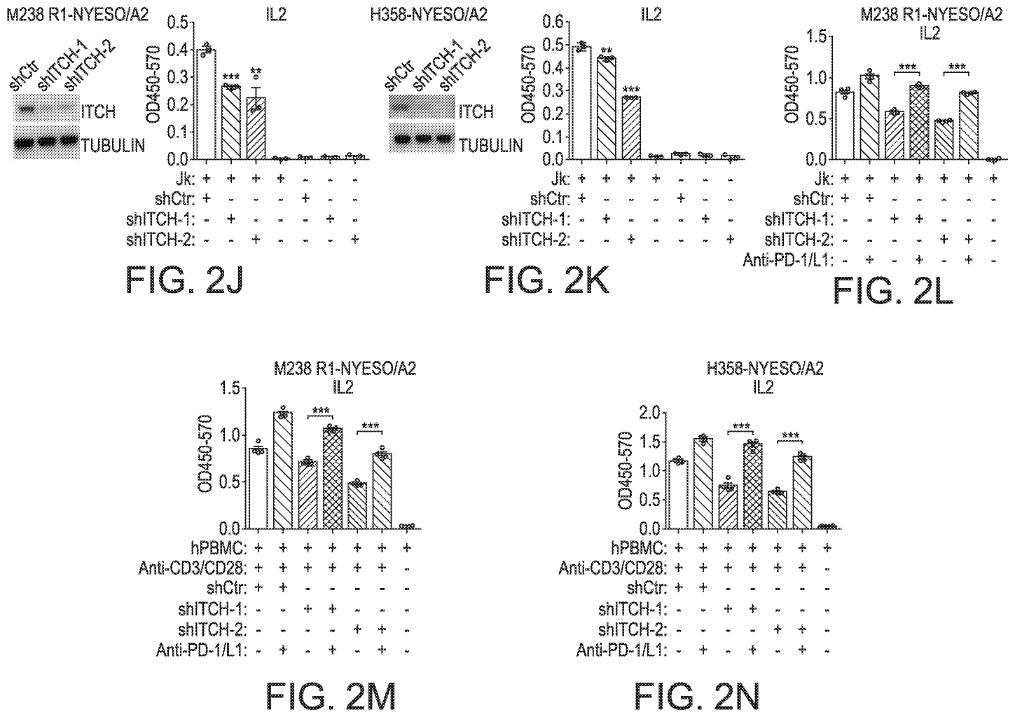

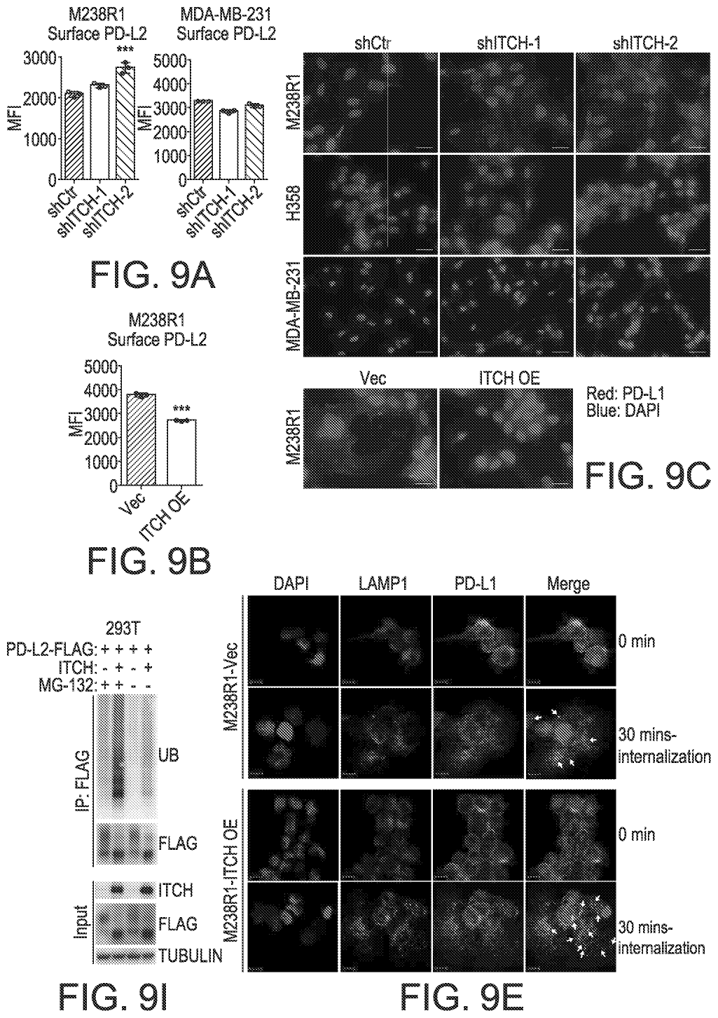

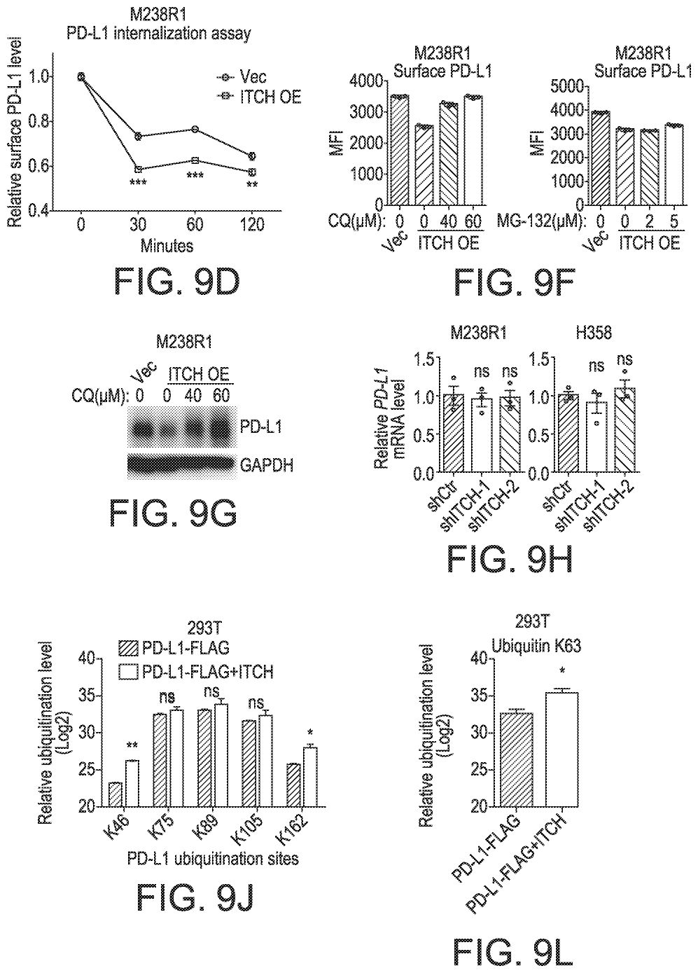

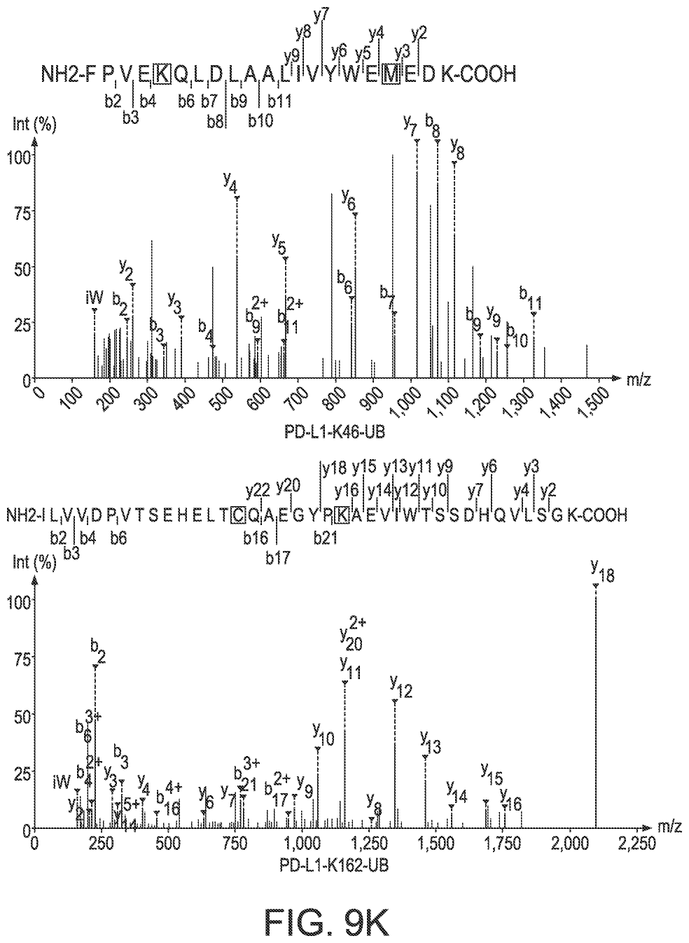

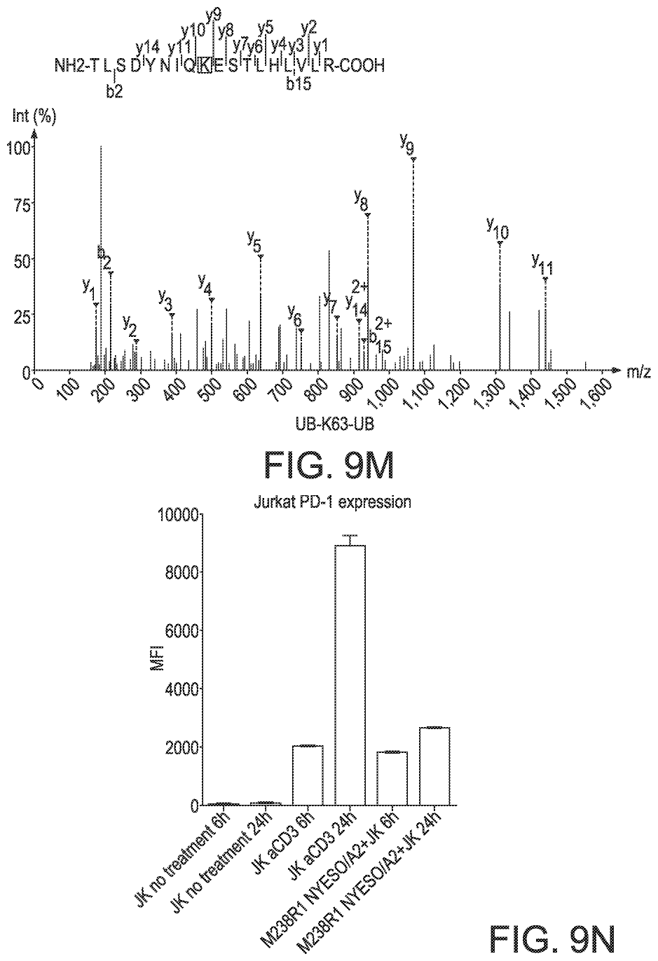

We then experimentally tested the hypothesis that ITCH, as an E3 ligase, down-regulates the total and cell-surface levels of PD-L1/L2 in cancer cells via ubiquitination. ITCH knockdown in M238 R1, H358, and MDA-MB-231 (a mesenchymal breast cancer cell line with abundant PD-L1 expression) by two independent shRNAs increased the total and cell-surface levels of PD-L1 (FIGS. 2A, 2B, 2C). ITCH knockdown increased the cell-surface level of PD-L2 to a lesser degree (versus PD-L1) in M238 R1 and elicited no effect in MDA-MB-231 (PD-L2 is undetectable in H358) (FIG. 9A). Conversely, ITCH over-expression in M238 R1 reduced PD-L1 and PD-L2 total or cell-surface levels (FIG. 2D; FIG. 9B). Measurements by Western blots and flow cytometry were confirmed by immunofluorescent visualization of PD-L1 with either ITCH knockdown or over-expression (FIG. 9C). Moreover, we showed that ITCH over-expression accelerated PD-L1 internalization from the cell surface (FIG. 9D) and enhanced PD-L1 co-localization with a lysosomal marker, LAMP-1 (FIG. 9E). Furthermore, treatment with a lysosomal inhibitor chloroquine (CQ), but not a proteasome inhibitor (MG-132), rescued down-regulation of cell-surface PD-L1 levels (FIG. 9F) and total PD-L1 levels (FIG. 9G) elicited by ITCH over-expression, suggesting that ITCH-dependent PD-L1 poly-ubiquitination marks PD-L1 for internalization followed by lysosomal degradation. Using real-time PCR, we showed that ITCH knockdown did not alter the PD-L1 mRNA levels (FIG. 9H). Using HEK 293T cells, we observed that co-transfection of ITCH with either PD-L1-FLAG (FIG. 2E) or PD-L2-FLAG (FIG. 9I) led to poly-ubiquitination of PD-L1 or PD-L2, respectively. Mass spectrometry analysis identified lysine 46 (K46) and K162 of PD-L1 as major ubiquitination sites caused by ITCH over-expression in 293T cells (FIGS. 9J, 9K). By analyzing ubiquitin branching or linkage patterns, we found that ITCH co-transfection with PD-L1 in 293T cells increased the level of K63-linked ubiquitin (FIGS. 9L, 9M), which is thought to control protein endocytosis, trafficking, and lysosomal degradation (16). Since PD-L1 and PD-L2 poly-ubiquitination was readily detectable in this over-expression system, we sought to detect poly-ubiquitination of endogenous PD-L1 in M238 R1 and H358. We found that endogenous PD-L1 was poly-ubiquitinated in both cancer cell lines tested (FIGS. 2F, 2G). Furthermore, knockdown of endogenous ITCH reduced PD-L1 poly-ubiquitination (FIGS. 2F, 2G), while over-expression of ITCH increased PD-L1 poly-ubiquitination (FIG. 2H). Thus, in cancer cells that display upregulated PD-L1 (and PD-L2) protein levels as the result of a spontaneous mesenchymal phenotype or BRAF^{V600MUT} inhibitor-induced mesenchymal transition/resistance, ITCH mediates poly-ubiquitination and reduces total and cell-surface protein levels of PD-L1 (and PD-L2).

### Tumor-Intrinsic ITCH Loss-of-Function Antagonizes T Cells Via PD-L1 Stabilization

To address whether the ITCH-PD-L1 physical and functional interaction constitutes a signaling axis that regulates cancer antigen-specific T-cells, we established a co-culture assay. Using lentiviral stable transduction, we engineered M238 R1 and H358 to express the cancer testis antigen, NY-ESO-1, fused with HLA-A2 and B2M. We then used a Jurkat T cell line (abbreviated as Jk) engineered with a T-cell receptor (TCR) clonotype (1G4) specific to NYESO-HLA-A2 (FIG. 2I) (17). The fusion of the NYESO peptide with a flexible linker, b2 microglobulin, another flexible linker, and then the heavy chain of HLA-A2 make up a single-chain trimer that enhances peptide antigen occupancy and presentation to CD8⁺ T-cells (18). We first confirmed that Jk T cells up-regulated PD-1 expression after treatment with anti-CD3 antibody or after antigen-specific stimulation by co-culture with MAPKi-resistant melanoma cells (FIG. 9N). Then, we observed that co-culture of M238 R1-NYESO/A2 or H358-NYESO/A2 with Jk T-cells, but not cancer cells or Jk T-cells alone, resulted in IL-2 secretion (FIGS. 2J to 2L). Importantly, ITCH knockdown, which up-regulated cancer cell-surface levels of PD-L1 and PD-L2, reduced IL-2 secretion (FIGS. 2J to 2L). Moreover, co-treatment with PD-1-plus PD-L1 blocking antibodies completely reversed this drop of IL-2 secretion (FIG. 2L), indicating that ITCH regulates CD8⁺ T-cell activation via ITCH-regulated PD-L1 (and PD-L2) levels. We confirmed this result by separate co-culture assays using, instead of Jk T cells, primary human peripheral blood mononuclear cells (PBMCs) activated by anti-CD3 and anti-CD28 antibodies (FIGS. 2M, 2N).

### ITCH Loss-of-Function Accelerates MAPKi-Resistance by Immune Evasion

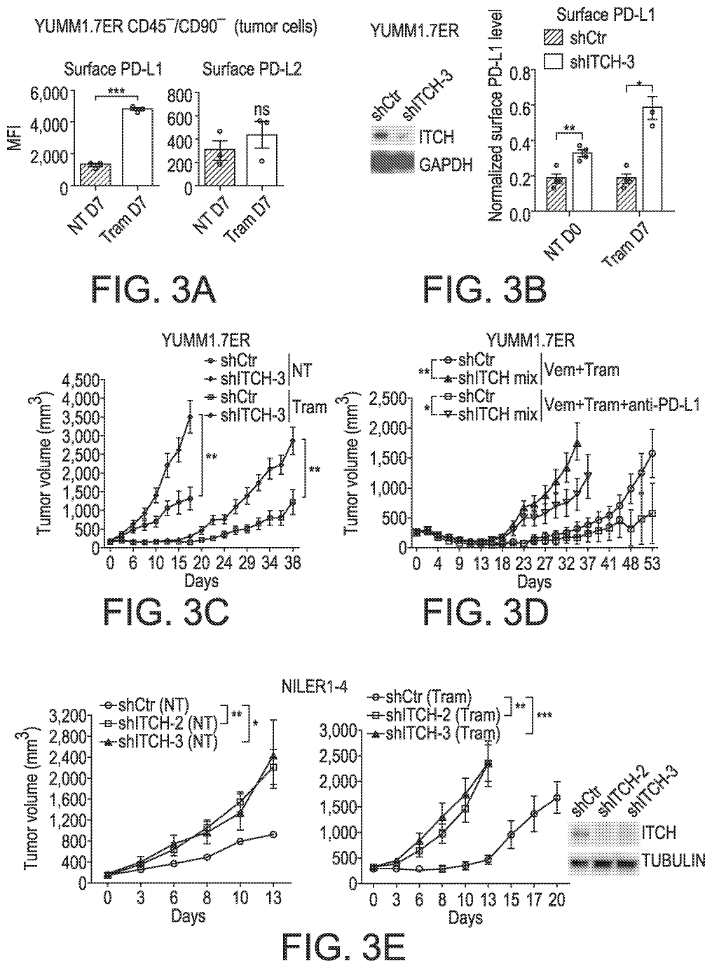

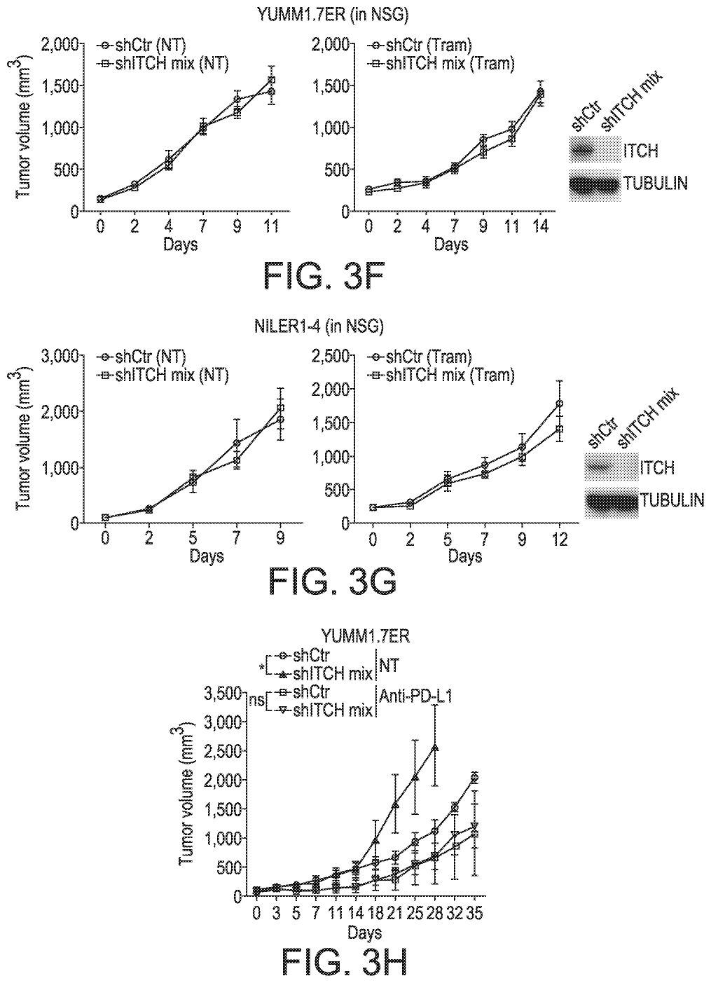

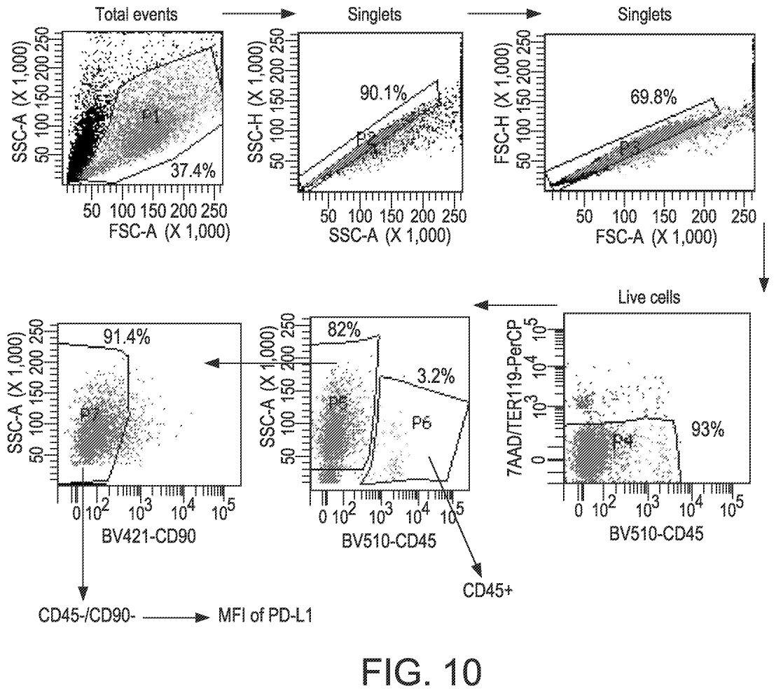

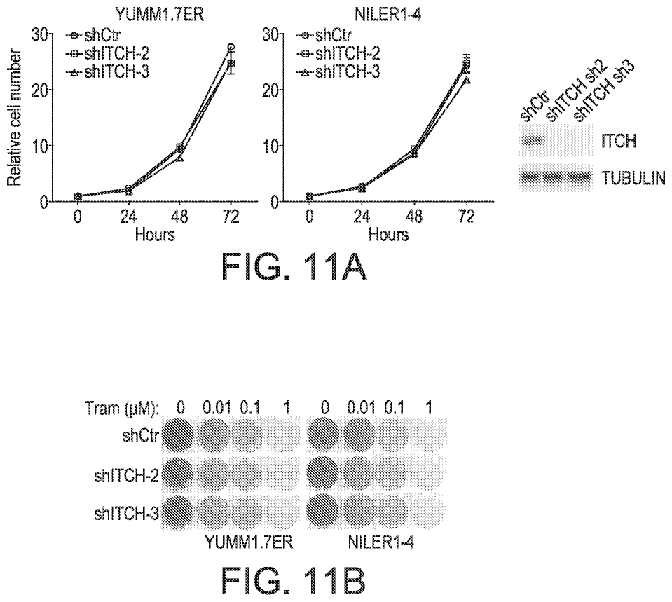

We then evaluated the functional impact of tumor cell-intrinsic ITCH in the context of an immune microenvironment in vivo and of therapy-induced PD-L1 upregulation in mouse tumor cells. We used a syngeneic Braf^{V600E} murine melanoma model (YUMM1.7ER) with high mutational burden caused by UV-mutagenesis (19). We first showed that 7-days of MEK inhibitor (MEKi) (trametinib at 1 mg/kg/d) treatment in vivo upregulated tumor cell-surface PD-L1 protein (FIG. 3A; FIG. 10). In this early window of therapy in vivo, we also tested the impact of Itch knockdown on tumor cell-surface levels of PD-L1 (FIG. 3B). We observed that, without treatment, ITCH knockdown upregulated the tumor cell-surface levels of PD-L1, to a level similar to that induced by 7-days of trametinib treatment. Moreover, ITCH knockdown in trametinib-treated tumors further upregulated PD-L1 on the tumor cell surface. Thus, in mouse melanoma cells in vivo, both basal (i.e., no treatment or NT) and therapy-induced levels of tumor cell-surface PD-L1 are subject to ITCH control. Importantly, ITCH knockdown accelerated basal tumor growth, which is consistent with lower ITCH levels being associated with worse survival in patients with cutaneous melanoma (FIG. 1H), as well as the onset of acquired resistance to MEK inhibition (FIG. 3C). Since the standard-of-care MAPKi treatment for patients with BRAFV600^{MUT} melanoma is a combination of BRAF^{V600MUT} and MEK inhibitors and since the triplet of BRAFV600^{MUT} and MEK inhibitors plus anti-PD-L1 has been approved clinically (20), we evaluated the effect of tumor cell-intrinsic ITCH knockdown on the onset of acquired resistance (FIG. 3D). ITCH knockdown also accelerated the development of acquired resistance to doublet and triplet therapies (FIG. 3D). To validate these tumor growth and therapy resistance phenotypes, we used another syngeneic murine melanoma model driven by Nras^{Q61R} (NILER1-4) with high mutational burden caused by UV-mutagenesis (12). In accordance with findings in Braf^{V600CE} melanoma, we found that ITCH knockdown accelerated both tumor growth without treatment and the development of trametinib (3 mg/kg/d) resistance (FIG. 3E). The tumor growth and therapy resistance phenotypes resulting from ITCH knockdown are dependent on T. B or NK cells, as we observed no differences in the growth curves of either YUMM1.7ER or NILER 1-4 tumors, with or without trametinib treatment (1 or 3 mg/kg/d, respectively), in NOD scid gamma (NSG) mice (FIGS. 3F and 3G). In addition, the tumor growth and therapy resistance phenotypes resulting from ITCH knockdown are not tumor cell-intrinsic, as we observed no differences in the short-term and long-term growth rates of YUMM1.7ER or NILER1-4 cell lines in vitro without or with trametinib treatment (FIGS. 11A, 11B). Since tumor cell-intrinsic ITCH knockdown accelerated tumor growth through tumor-cell extrinsic or immune mechanisms, we tested whether this effect is due to induction of tumor cell-surface PD-L1 levels in vivo. To do so, we treated mice with an anti-PD-L1 antibody (without co-treatment with a MEKi) (FIG. 3H) to neutralize PD-L1 tumor cell-surface upregulation mediated by ITCH knockdown (FIG. 3B). As expected, ITCH knockdown and anti-PD-L1 treatment respectively accelerated and decelerated YUMM1.7ER tumor growth (FIG. 3H). Importantly, in the presence of both tumor cell-intrinsic ITCH knockdown and anti-PD-L1 treatment, we abolished the ability of ITCH knockdown to accelerate tumor growth in an immune competent host (FIG. 3H).

### Tumor-Intrinsic ITCH Loss-of-Function Creates a Pro-Tumorigenic Immune Microenvironment

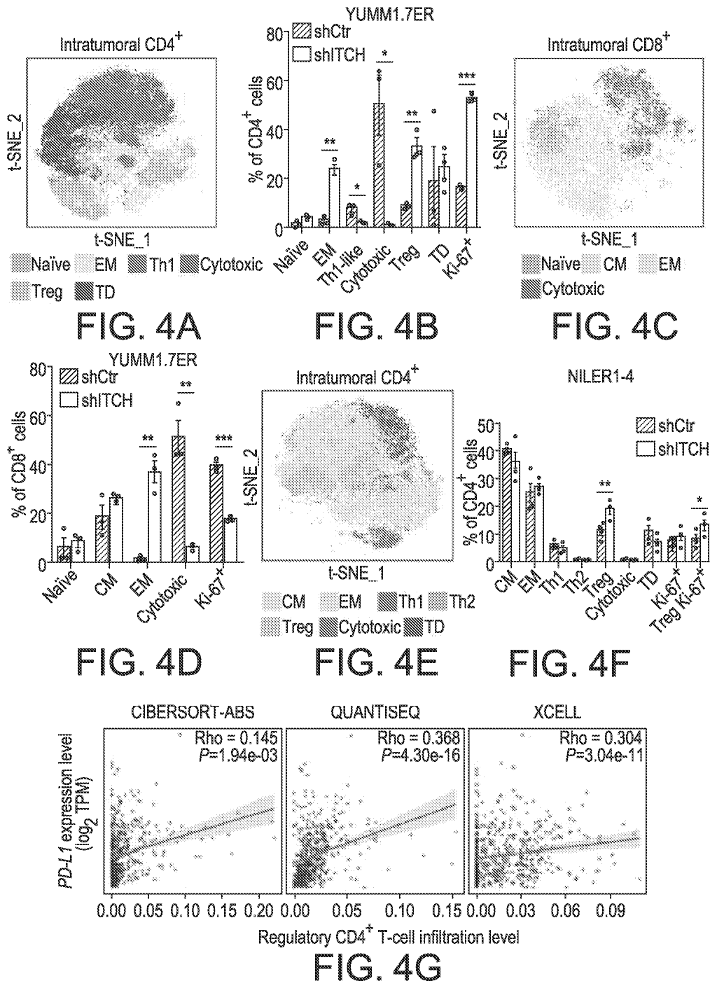

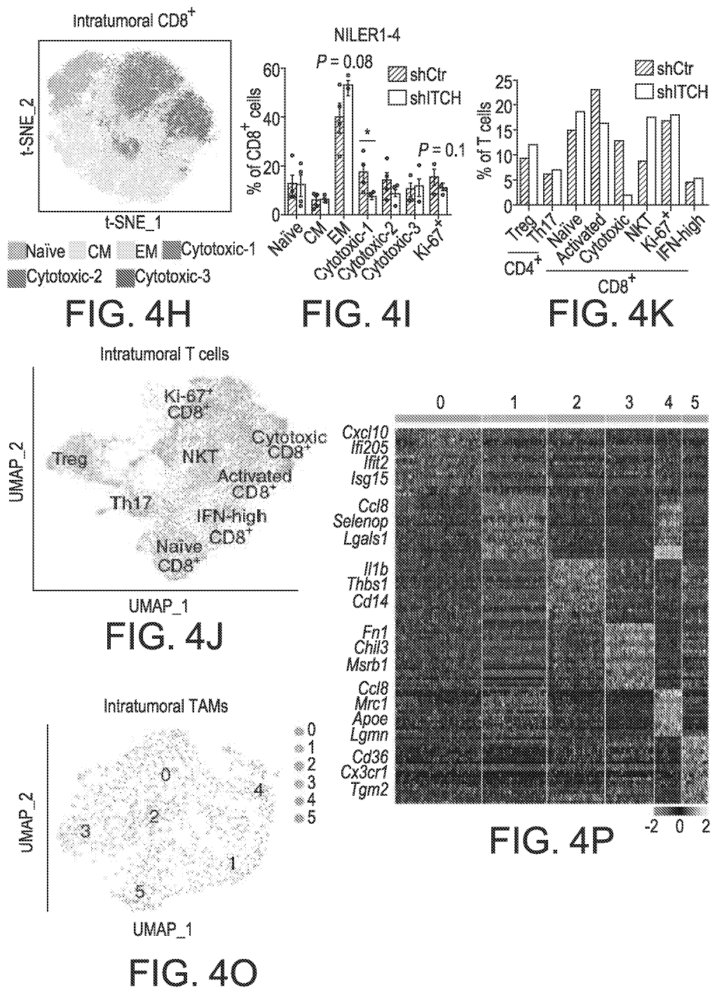

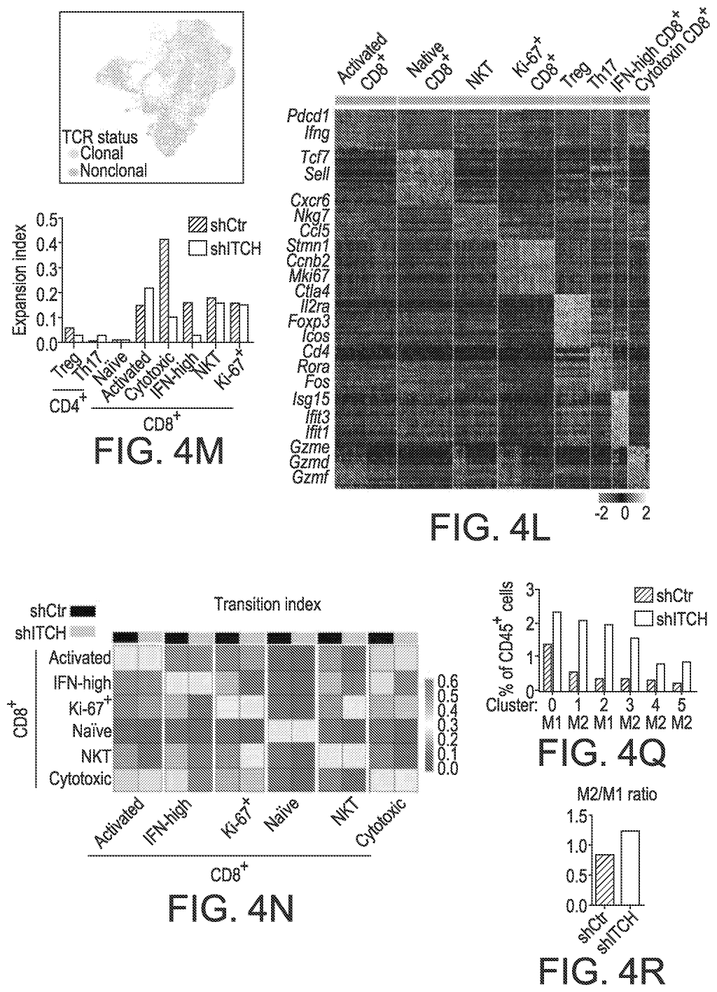

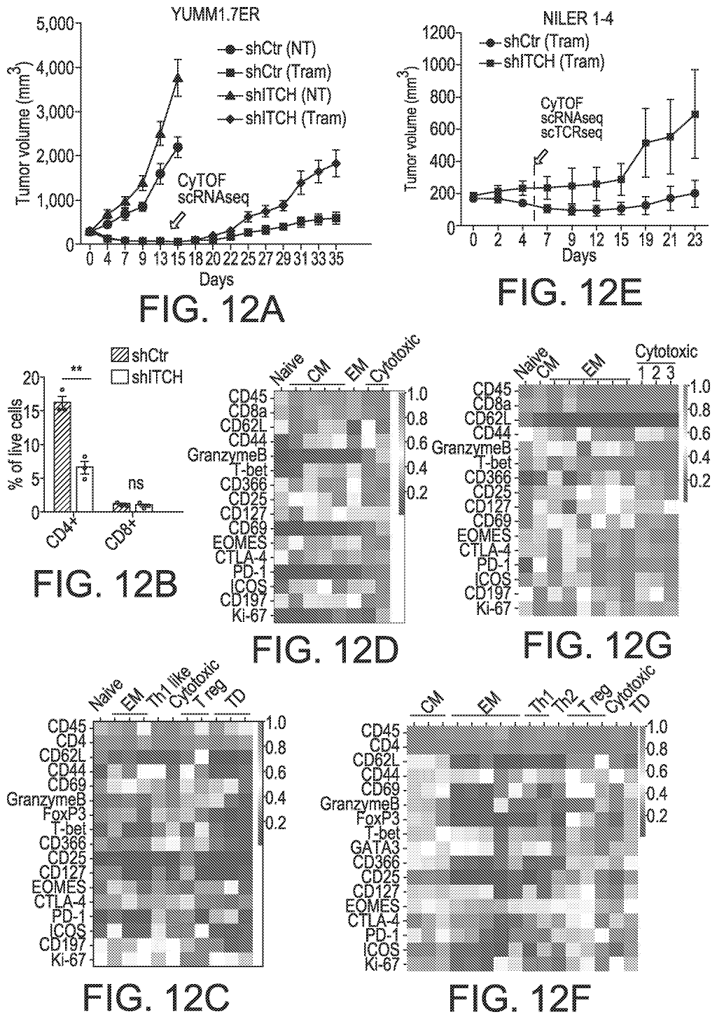

To investigate ITCH knockdown-induced changes in the tumor immune microenvironment, we profiled YUMM1.7ER shCONTROL and ITCH-knockdown tumors after 14 days of trametinib treatment (1 mg/kg/d) by performing cytometry by time of flight (CyTOF) (FIG. 12A). Analysis of CyTOF data from the dissociated tumors (n=3 per group) showed that ITCH-knockdown (vs. shCONTROL) tumors harbored less CD4⁺ T cell infiltration (FIG. 12B). Sub-clustering analysis of intratumoral CD4⁺ T cells revealed that ITCH-knockdown tumors contained higher fractions of regulatory CD4⁺ T cells (TREG, CD4⁺FOXP3⁺) and effector/effector memory CD4⁺ T cells (EM, CD4⁺ CD62L CD44⁺) but lower fractions of cytotoxic CD4⁺ T cells (CD4⁺Granzyme B⁺) and Th1-like CD4⁺ T cells (CD4⁺ T-bet⁺) (FIGS. 4A, 4B). TREG also showed the highest Ki-67 expression across different CD4⁺ T cell subclusters, indicating more proliferative regulatory T cells in ITCH-knockdown tumors early on-treatment (FIG. 12C). Analysis of intratumoral CD8⁺ T cells showed that ITCH-knockdown tumors harbored much fewer cytotoxic CD8⁺ T cells (CD8⁺Granzyme B⁺) but more effector/effector memory CD8⁺ T cells (EM, CD8⁺ CD62L CD44⁺). Also, the fraction of Ki-67⁺, proliferative CD8⁺ T cells was much lower in ITCH-knockdown (vs. shCONTROL) tumors (FIGS. 4C, 4D; FIG. 12D). We also used CyTOF to profile NILER1-4 shCONTROL vs. ITCH-knockdown tumors 5 days after trametinib (3 mg/kg/d) treatment (FIG. 12E). CyTOF analysis of dissociated NILER1-4 tumors (n=4 per group) also showed that ITCH-knockdown (vs. shCONTROL) tumors contained higher fractions of regulatory CD4⁺ T cells and proliferating regulatory CD4⁺ T cells (FIGS. 4E, 4F; FIG. 12F). Tumor or antigen presenting cells' expression of PD-L1 has been shown to convert naïve CD4⁺ or T helper 1 cells to regulatory or suppressor CD4⁺ T cells (21,22). Consistently, from an analysis of 471 clinical cutaneous melanoma samples using TIMER2.0, intratumoral PD-L1 expression was positively correlated with regulatory CD4⁺ T cell infiltration (FIG. 4G). Analysis of intratumoral CD8⁺ T cells shows that ITCH-knockdown NILER 1.4 tumors harbored fewer cytotoxic CD8⁺ T cells in general and cluster 1 (Cytotoxic-1) in particular (FIGS. 4H, 4I, FIG. 12G). Also, the fraction of Ki-67⁺ CD8⁺ T cells trended lower in ITCH-knockdown (vs. shCONTROL) NILER1-4 tumors. Thus, CyTOF data derived from two murine melanoma models support the concept that tumor cell-expressed ITCH during MAPKi therapy suppresses the expansion of intratumoral regulatory CD4⁺ T cells and promotes the expansion of cytotoxic CD8⁺ T cells. These immune cellular effects are consistent with the impacts of ITCH knockdown in up-regulating PD-L1 tumor cell-surface levels in MAPKi-treated melanoma (FIGS. 1 to 3) and in accelerating the development of MAPKi resistance only in immune-competent hosts (FIG. 3)

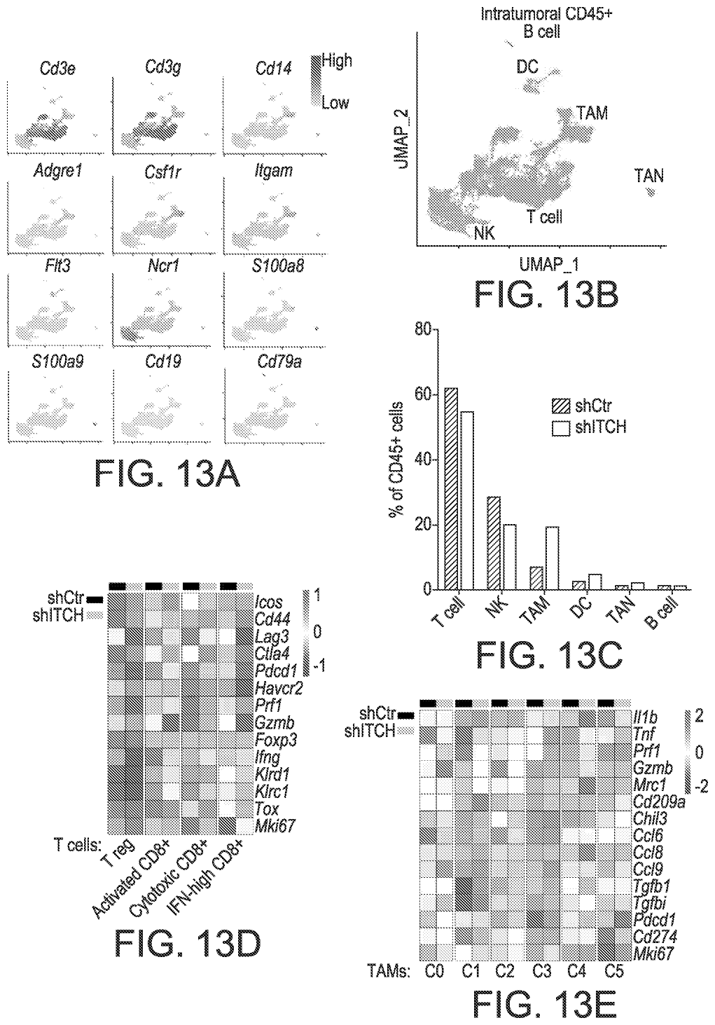

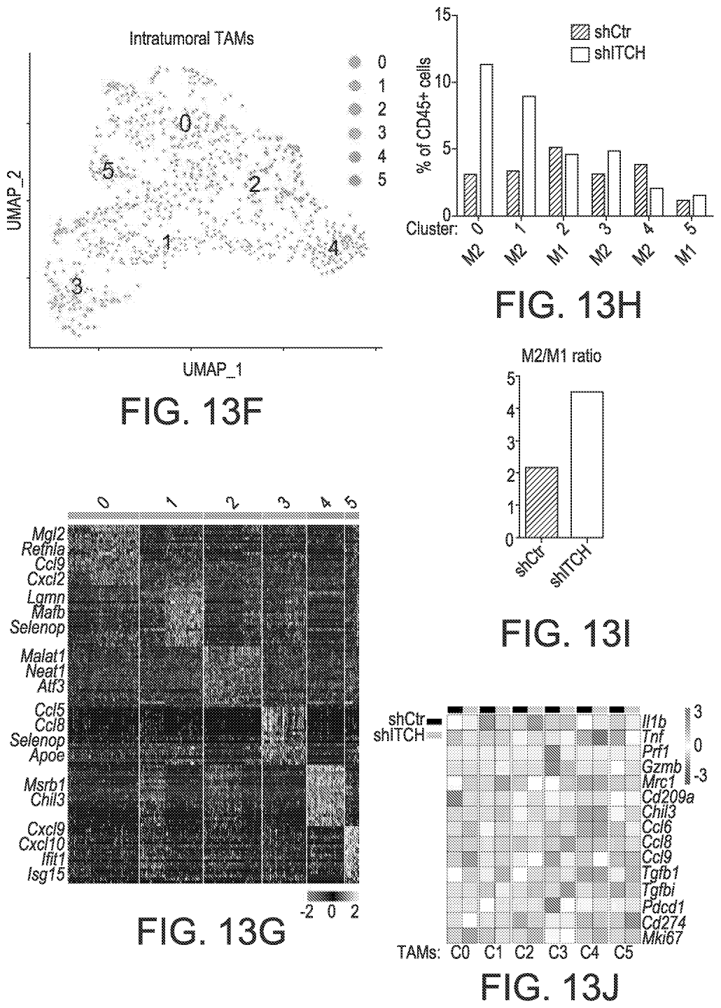

To dissect the impact of ITCH knockdown on the tumor immune microenvironment comprehensively, we performed analysis of single-cell RNA sequencing (scRNA-seq) data coupled to single-cell T-cell receptor sequencing (scTCR-seq) data to evaluate deeper immune phenotypic alterations in shCONTROL vs. ITCH-knockdown NILER1-4 tumors early on MEKi treatment (FIG. 12E). Four tumors per group were dissociated and combined into one sample to sort for the CD45⁺ population. A total of 15,532 CD45⁺ cells were identified in scRNA-seq data analysis, and immune cell clusters were annotated by key lineage markers (FIGS. 13A, 13B). ITCH-knockdown tumors showed higher fractions of tumor-associated macrophages (TAMs) and neutrophils (TANs) but a lower fraction of T cells among CD45⁺ cells (FIG. 13C). Sub-clustering of the T-cell population identified 8 subpopulations based on differentially expressed genes (FIGS. 4J to 4L). Consistent with CyTOF-based findings. ITCH-knockdown tumors harbored a higher fraction of regulatory CD4⁺ T cells (cluster 4) but lower fractions of activated and cytotoxic CD8⁺ T cells (FIGS. 4K, 4L). Moreover, regulatory CD4⁺ T cells in ITCH-knockdown tumors were more proliferative and less exhausted with higher Mki67 and lower Lag3 expression (FIG. 13D). In addition, in ITCH-knockdown tumors, CD8⁺ T cells were less active and cytotoxic based on lower expression of ling. Pdcd1, Lag3, Prf1, Gzmb. KIrd1, and Kirc1 (FIG. 13D). From scTCR-seq data analysis, we identified a total of 1,724 TCR clonotypes with unique a and b chain pairs. 424 of these clonotypes were represented by two or more cells, which defined 3,248 clonal T cells (FIG. 4M). We then analyzed clonal expansion of different T-cell subpopulations and observed that shiICH (vs. shCONTROL) tumors harbored less expansion of cytotoxic and IFN^{high} CD8⁺ T cells (FIG. 4M). Additional analysis revealed a consistent pattern in shiICH (vs. shCONTROL) tumors of lower transition indices between pairs of CD8⁺ T-cell subpopulations, indicating that reduced ITCH expression in tumor cells blunted phenotypic conversions among functional T-cell subsets and blocked naïve T-cell activation (FIG. 4N).

Since the TAM population nearly tripled in size with ITCH knockdown, we defined 6 sub-clusters based on differential gene expression (FIGS. 4O, 4P). We identified cluster 0 and 2 as M1-like TAMs because of high expression of pro-inflammatory cytokines and/or M1 markers such as Cxcl10, Ifi205, Il1b and Thbs1. Cluster 1, 3, 4, and 5 were identified as M2-like TAMs, since they expressed highly anti-inflammatory or pro-tumorigenic cytokines and/or M2 markers such as Ccl8, Selenop, Fn1, Chil3, Mrc1, Apoe, Lgmn, Tgm2, etc. (FIG. 4P). Importantly, we found that ITCH-knockdown tumors not only contained higher fractions of every TAM subpopulation (FIG. 4Q) but also a higher M2 to M1 ratio (FIG. 4R). Furthermore, analysis of gene expression levels showed that the TAM subpopulations in ITCH-knockdown tumors tend to express lower levels of pro-inflammatory cytokines such as Il1b, Tnf and cytotoxic genes such as Gzmb and Prf1 but higher levels of M2 macrophage markers such as Mrc1, Cd209a and Chil3 as well as pro-tumorigenic cytokines such as Ccl6, Ccl8, Ccl9, Tgfb1 and Tgfbi (FIG. 13E). To validate modulation in TAMs by ITCH knockdown early during MAPKi treatment in the YUMM1.7ER model, we analyzed scRNA-seq data derived from whole tumors (i.e., non-CD45+ sorted) (FIG. 12A). We were able to detect 886 TAMs in total. Re-clustering of the TAM population identified 6 subclusters (FIGS. 13F, 13G). We defined clusters 2 and 5 as M1-like TAMs based on their expression of pro-inflammatory cytokines and M1-polarization related genes such as Cxcl9, Cxcl10, Malat1 and Neat1. Cluster 0, 1, 3 and 4 were identified as M2-like TAMs based on the expression of anti-inflammatory cytokines or pro-tumorigenic cytokines and M2 markers such as Mgi2. Ccl9, Cxcl2, Lgmn, Selenop, Ccl5, Ccl8, Apoe, Chil3, etc. (FIG. 13G). Consistent with findings from the NILER1-4-derived scRNA-seq data, ITCH knockdown resulted in much higher levels of M2-like TAMs (cluster 0, 1, 3) within early on MAPKi-treated tumors (FIG. 13H). The ratio of M2 to M1 TAMs in ITCH-knockdown tumors was ˜two-fold higher than in shCONTROL tumors (FIG. 13I). Finally, TAM clusters in ITCH-knockdown tumor tended to express lower levels of pro-inflammatory cytokines (Il1b, Tnf) and cytotoxic gene (Prf1, Gzmb) but higher levels of the M2 macrophage marker Mrc1 and the pro-tumorigenic cytokines (Ccl6, Ccl8, Ccl9) (FIG. 13J).

### ITCH Suppresses MAPKi-Resistance by PD-L1 Down-Regulation and CD8⁺ T-Cell Up-Regulation

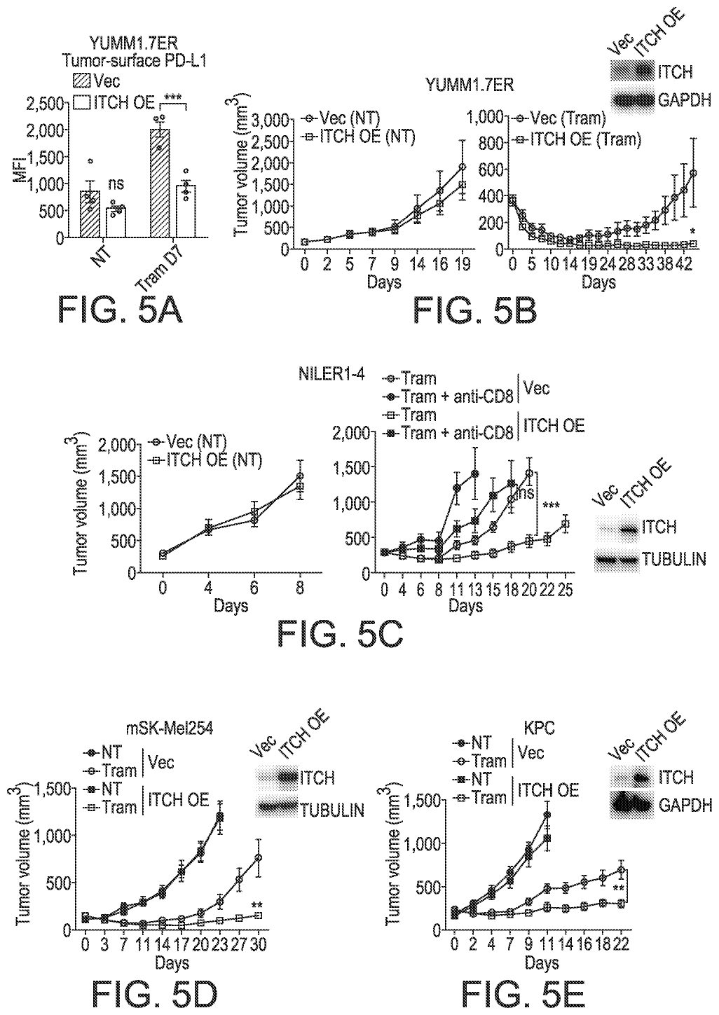

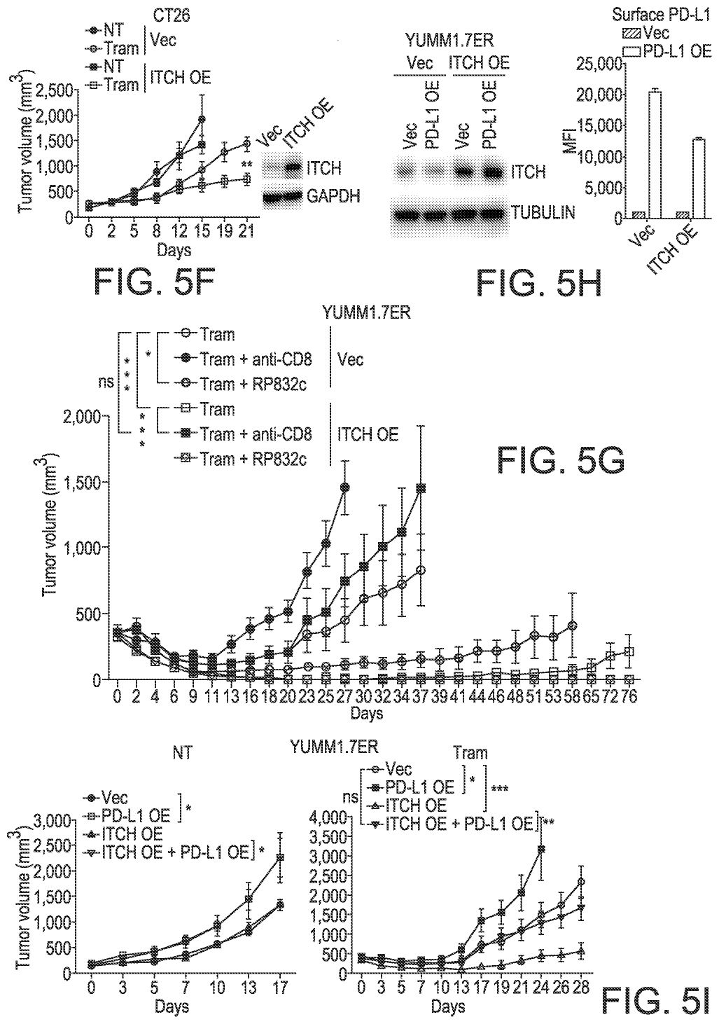

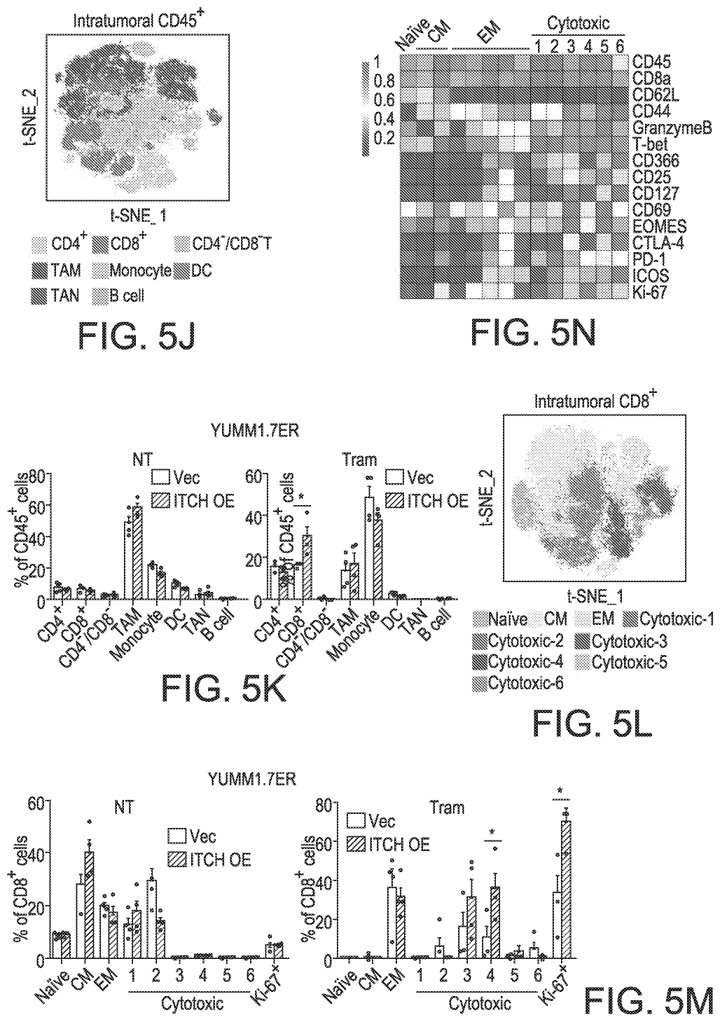

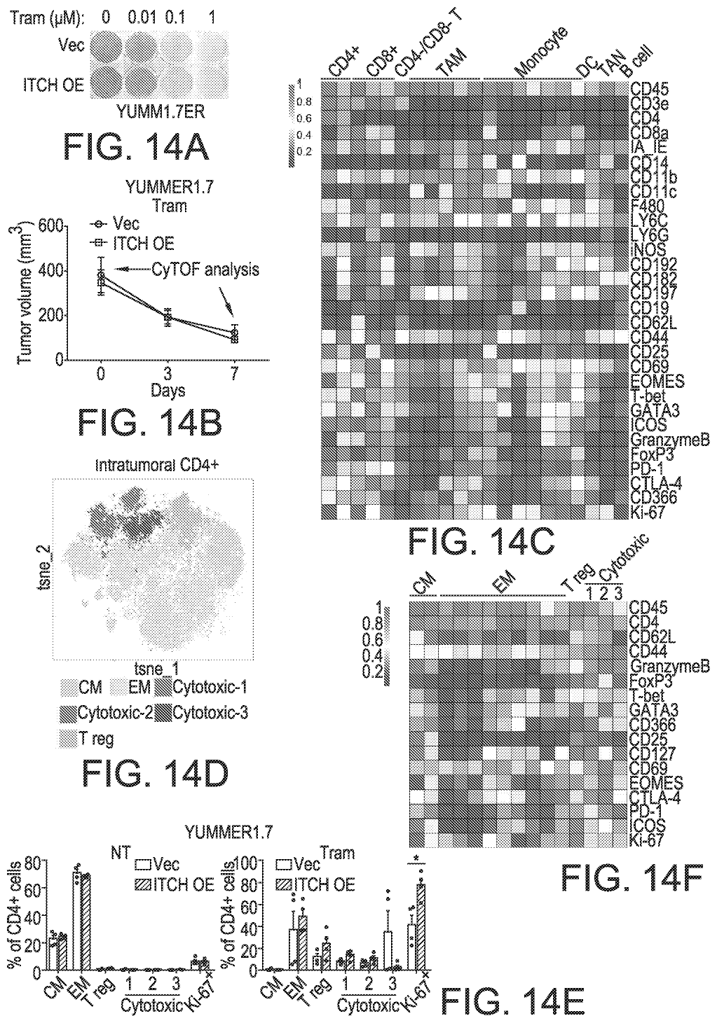

Based on the above findings, we hypothesized that ITCH over-expression in tumor cells should suppress acquired MAPKi resistance. We engineered YUMMER1.7 cells to express empty vector (Vec) or harbor ITCH over-expression (OE); these two sub-lines grew at indistinguishable rates in vitro, with or without MEK inhibitor (trametinib) treatment (FIG. 14A). After implanting these two cell lines into syngeneic mice and allowing tumors to grow exponentially (reaching ˜400 mm³), flow cytometry analysis of tumors with no treatment versus trametinib treatment (0.45 mg/kg/day×7 days) showed that, as expected (FIG. 3A), in vivo MEK inhibition induced the tumor cell-intrinsic surface-level of PD-L1 and, importantly, ITCH over-expression reduced the tumor cell-intrinsic surface-level of PD-L1 to the level observed in treatment-naïve tumors (FIG. 5A). Furthermore, ITCH over-expression in YUMMER1.7 tumor cells did not inhibit tumor growth in the absence of MAPKi treatment (FIG. 5B), likely because basal PD-L1 expression is already low (FIG. 5A). However, as hypothesized, after MAPKi treatment of established (˜400 mm³) tumors, ITCH over-expression strongly suppressed resistance development (FIG. 5B). At the last follow-up (day 60) of MAPKi-treated tumors with ITCH over-expression, we did not observe any case of acquired resistance, with six of ten tumors displaying complete responses. We tested this hypothesis further in 3 additional syngeneic tumor models with variable levels of trametinib responsiveness (13); these included NIL1ER1-4 (Nras^{MUT} melanoma), mSK-Mel254 (Nf1-melanoma), and KPC (Kras^{MUT} PDAC) (FIGS. 5C to 5E). In every additional model tested, tumor cell-intrinsic ITCH over-expression suppressed acquired MAPKi-resistance.

Based on prior in vitro and in vivo analyses of an ITCH-PD-L1-T-cell regulatory axis (FIGS. 2 to 4), we also hypothesized that suppression of acquired MAPKi-resistance by ITCH over-expression requires CD8⁺ T cells. Importantly, in both syngeneic models tested, systemic CD8 neutralization and depletion of CD8⁺ T cells completely rescued or reversed suppression of MEK inhibitor resistance caused by tumor-intrinsic ITCH over-expression (FIGS. 5C, 5F). Moreover, since tumor cell-intrinsic ITCH knockdown led to M2 TAM polarization (FIG. 4; FIG. 13), we tested whether targeting M2-like TAMs by a peptide agonist of CD206 (RP832c) could suppress trametinib resistance (13, 23, 24). Although it was difficult to discern an added resistance-suppressive effect of RP832c on ITCH over-expressing tumors, RP832c treatment (dosed daily over the first 7 days) phenocopied the resistance-suppressive effect of ITCH over-expression (FIG. 5F). Prior analyses supported the notion that ITCH modulates T-cell functions in vitro (FIGS. 2J to 2N) or tumor growth in immune competent hosts (FIG. 3H) via regulating PD-L1. To support this concept further, we hypothesized that tumor cell-intrinsic over-expression of PD-L1 on top of ITCH over-expression (FIG. 5G) in YUMM1.7ER cells would nullify the resistance-suppressive phenotype of ITCH over-expression. Indeed, while ITCH over-expression suppressed acquired trametinib resistance, combined over-expression of ITCH and PD-L1 reversed the ITCH-mediated resistance-suppressive phenotype and restored the resistance developmental kinetics to that observed without ITCH over-expression (FIG. 5H).

Next, we performed CyTOF to analyze the tumor immune microenvironments before and after MAPKi treatment of mice-bearing tumors without or with tumor-intrinsic ITCH over-expression (n=4 tumors per group) (FIG. 14B). CD45⁺ immune cells were gated out for sub-clustering analysis according to their expression of immune cell lineage markers. Eight different cell populations were identified, and we found the fraction of CD8⁺ T cells in the CD45⁺ cell population under trametinib treatment was higher in tumors with ITCH over-expression. (FIGS. 5I, 5J; FIG. 14C). Sub-clustering analysis of the CD8⁺ T-cell population identified 9 distinct functional sub-populations. On trametinib treatment (but not before treatment), ITCH over-expression enhanced CD8⁺ T-cell proliferation (fraction of Ki-67⁺ cells) and increased the fraction of the most proliferative, cytotoxic CD8⁺ T cells (cluster cytotoxic-4, Ki-67 highest) (FIGS. 5K to 5L). Sub-clustering analysis of the CD4⁺ T-cell population showed 6 different sub-populations, and ITCH over-expression also increased CD4⁺ T-cell proliferation (fraction of Ki-67⁺ cells) (FIGS. 14D to 14F). These CyTOF findings mirror our previous findings with tumor cell-ITCH knockdown (FIG. 4) and the positive clinical correlation between ITCH expression and CD8⁺ T cell-infiltration and FIG. 8C). These CyTOF findings also support the in vivo phenotype of tumor-intrinsic ITCH over-expression, namely, MAPKi resistance-suppression that is dependent on PD-L1 down-regulation (FIG. 5H) and CD8⁺ T cells (FIGS. 5C, 5F).

Identification of a Small Molecular ITCH Activator that Suppresses Acquired MAPKi-Resistance

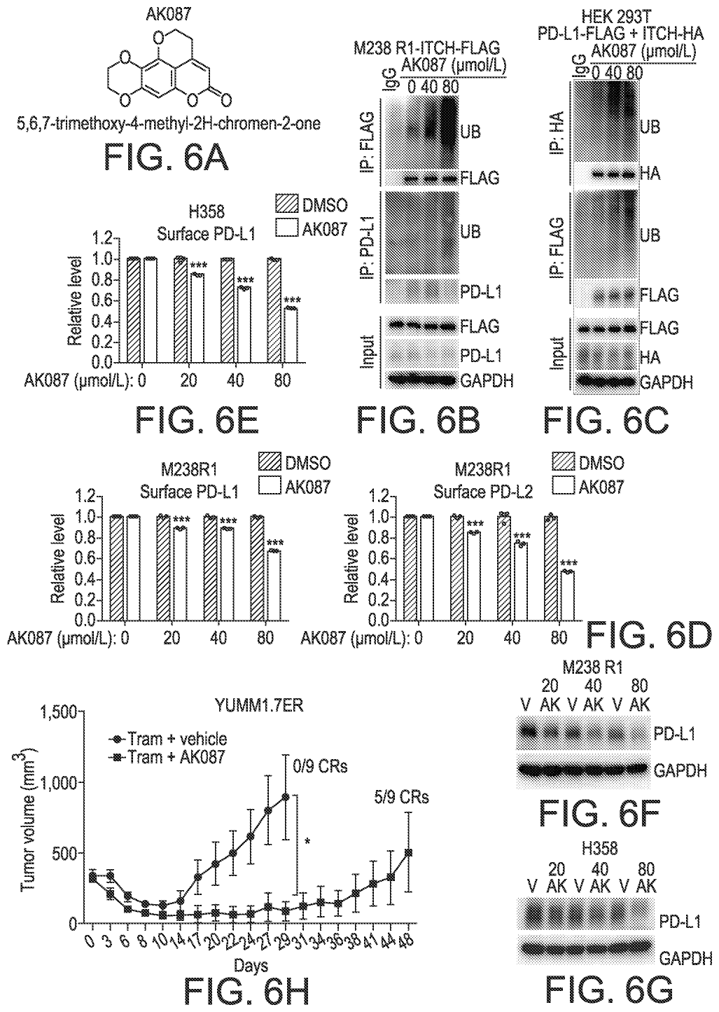

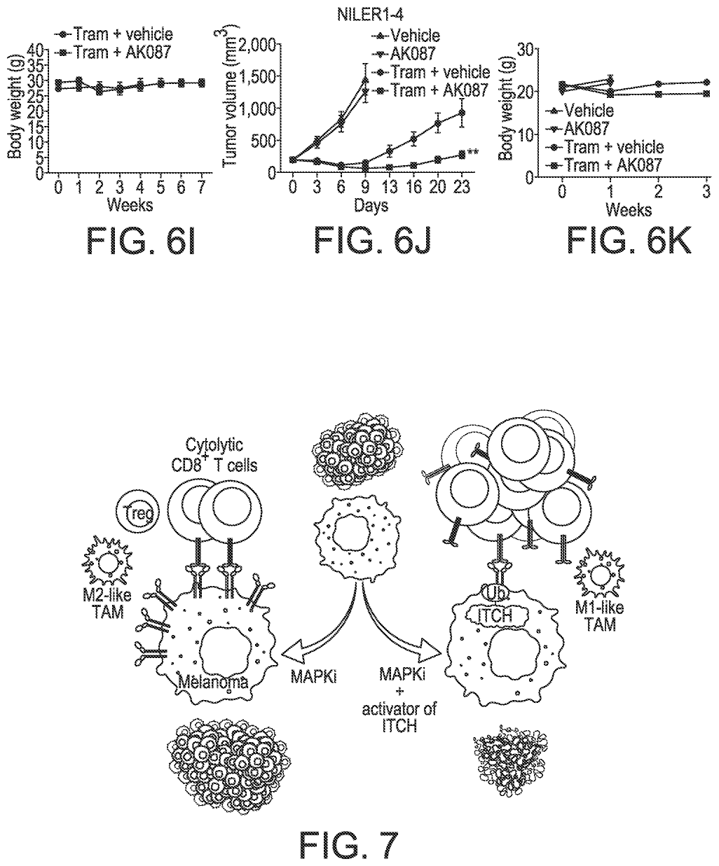

We then sought to provide in vivo proof-of-principle data supportive of the translational potential of our findings above by identifying an ITCH activator. First, we queried a small molecule screen intended to identify ITCH inhibitors (25) for potential ITCH activators. One compound, AK087 (FIG. 6A), displayed an ability to enhance (140%) the auto-ubiquitination of ITCH in an assay of reconstituted E1, E2, E3 (ITCH), and ubiquitin (tagged with horseradish peroxidase) recombinant proteins. AK087 dose-dependently enhanced the poly/auto-ubiquitination of ITCH-FLAG expressed in a human melanoma cell lines adapted to BRAF inhibition (M238 R1), while enhancing the poly-ubiquitination of endogenous PD-L1 (FIG. 6B). In human 293T cells co-expressing PD-L1-FLAG and ITCH-HA, we observed similar findings (FIG. 6C). Moreover, in human melanoma cells with upregulated surface-PD-L1/L2 (M238 R1) and human lung cancer cell line with upregulated surface-PD-L1 (H358), AK087 dose-dependently reduced the tumor cell-surface (FIGS. 6D, 6E) and total (FIGS. 6F, 6G) of PD-L1/L2. Lastly and importantly, AK087 co-treatment (dosed subcutaneously at 10 mg/kg/day for 17 days) with trametinib suppressed acquired MAPKi-resistance in mice bearing YUMM1.7ER melanoma tumors, which was associated with 5 of 9 complete responses (versus 0 of 9 complete responses without AK087 co-treatment) and no appreciable body weight loss or superficial evidence of toxicities (FIGS. 6H, 6I).

### Discussion

Harnessing antitumor immunity based on release of a key immune checkpoint interaction between PD-L1 and PD-1 has revolutionized oncology therapy. PD-L1 expression level in tumor cells is highly dynamic and regulated as concerted responses to alterations in tumor-intrinsic cell states and immunologic cues. Regulation at post-translational levels can occur via ubiquitination, deubiquitination, compartmentalization, glycosylation, palmitoylation, and phosphorylation. The current study supports a model (FIG. 7) in which the E3 ligase ITCH mediates poly-ubiquitination of PD-L1 and down-regulates tumor cell-surface PD-L1 levels (via ubiquitin-directed lysosomal degradation) in MAPKi-adapted melanoma cells and in potentially other biologic contexts such quasi-mesenchymal tumor cell-states that contribute to therapy resistance and metastatic potential. Our prior study (3) has demonstrated PD-L1/L2 up-regulation as a recurrent and early MAPKi response in clinical melanoma as well as in cell line and in vivo syngeneic models of melanoma. By down-regulating surface PD-L1 in MAPKi-treated melanoma, tumor cell-intrinsic ITCH promotes tumor immune surveillance by CD8⁺ T cells and may also ameliorate immune-suppressive microenvironmental features such as pro-tumorigenic macrophages and TREG differentiation (FIG. 7). Hence, we nominate tumor cell-surface PD-L1 stability and tumor-intrinsic ITCH activity as MAPKi co-targets. We propose to develop pharmacologic strategies to destabilize cell-surface PD-L1 or to activate ITCH function within melanoma cells in order to develop combinatorial strategies to prevent adaptive immune resistance and, thereby, acquired MAPKi resistance.

What immunologic factors dictate the durability of clinical responses to MAPKi remain ill-defined. The manners by which syngeneic murine melanoma, in response to MAPKi therapy, undergoes “immunogenic” cell death (26) and evades antitumor immunity (this study) directly regulate the durability of MAPKi responses, as do tumor mutational burden (this study and in our prior study of Nras^{MUT} melanoma (12). MAPKi therapy in BrafV500^{MUT} melanoma (without mutational burden) elicits pyroptotic cell death and tumor necrosis. Release of chromatin protein HMGB1 by necrotic cells can trigger pro-tumorigenic inflammation associated with TAMs (27,28). Thus, our additional findings that ITCH loss-of-function leads to M2-like TAM polarization and that pharmacologic targeting of M2-like TAMs phenocopies ITCH gain-of-function (in suppressing resistance) suggest an alternative immune-based strategy to enhance the durability of MAPKi responses. ITCH loss-of-function also induces the levels of TREG cells. As pharmacologic strategies targeting TREG cells advance to the clinic (29,30), the repertoire of immune-based strategies to enhance the durability of MAPKi responses may also expand.

The simultaneous combination of anti-PD-L1 with BRAFV600^{MUT} and MEK inhibitors (so-called “triplet” therapy), in early clinical data, appears beneficial and has been approved for patients with BRAFV600^{MUT} melanoma (20). Retrospective clinical data analysis and in vivo therapeutic modeling showed that a regimen of anti-PD-1/L1 (±anti-CTLA-4) lead-in before MAPKi combination augments the efficacy of triplet therapy by enhancing MAPKi durability (and overcoming innate resistance to immune checkpoint blockade) (13). Consistent with a pro-tumorigenic immune microenvironment elicited by depleting tumor cell expression of ITCH, this sequential regimen maximizes anti-tumorigenic immunity via remodeling a similar network of innate and adaptive immune cells that are operative during the early phase of MAPKi therapy.

A still maturing but promising area of cancer therapeutics development lies in proteolysis-targeting chimeras (PROTACs) and related approaches using immunomodulatory drugs that induce targeted protein degradation by the ubiquitin-proteasome pathway (31). PROTACs serve as bridges that bring together a protein being targeted for degradation and a non-native or non-physiologic E3 ligase. The Example provides a proof-of-concept of small molecular E3 ligase activation as an alternative approach to target a natural E3 substrate for degradation. MAPK inhibitors, although a success in oncology drug development, remains non-curative for a large portion of patients with BRAFV600^{MUT} cutaneous melanoma and experimental (due to limited monotherapy efficacy) for the majority of patients with MAPK-addicted cancer histologies. Thus, direct or indirect PD-L1 degraders should be co-developed with MAPK and, potentially, immune checkpoint inhibitor therapies.

### REFERENCES

1. Sharma P, et al. Cell 2017; 168:707-23
2. Dongre A, Weinberg R A. Nat Rev Mol Cell Biol 2019; 20:69-84
3. Song C, et al. Cancer Discov 2017; 7:1248-65
4. Hugo W. et al. Cell 2015; 162:1271-85
5. Hugo W, et al. Cell 2016; 165:35-44
6. Goodman A M, et al. JAMA Oncol 2018; 4:1237-44
7. Kataoka K, et al. Nature 2016; 534:402-6
8. Burr M L, et al. Nature 2017; 549:101-5
9. Huang X, et al. Cancer Immunol Res 2019; 7:1580-90
10. Mezzadra R, et al. Nature 2017:549:106-10
11. Zhang J, et al. Nature 2018; 553:91-5
12. Hong A, et al. Cancer Discov 2021; 11:714-35
13. Wang Y, et al. Cancer Cell 2021
14. Nazarian R et al. Nature 2010; 468:973-7
15. Li J, et al. Nat Methods 2013; 10:1046-7
16. Erpapazoglou Z, et al. Cells 2014; 3:1027-88
17. Bethune M T, et al. Elife 2016; 5
18. Hansen T, et al. Curr Protoc Immunol 2009; Chapter 17: Unit17 5
19. Wang J, et al. Pigment Cell Melanoma Res 2017; 30:428-35
20. Gutzmer R, et al. Lancet 2020; 395:1835-44
21. Amarnath S, et al. Sci Transl Med 2011; 3: 111ra20
22. Francisco L M, et al. J Exp Med 2009; 206:3015-29
23. Ghebremedhin A, et al. bioRxiv 2020
24. Jaynes J M, et al. Sci Transl Med 2020; 12
25. Rossi M, et al. Cell Death Dis 2014; 5:e1203
26. Erkes D A, et al. Cancer Discov 2020; 10:254-69
27. Scafidi P, et al. Nature 2002; 418:191-5
28. Vakkila J, et al. Nat Rev Immunol 2004; 4:641-8
29. Solomon I, et al. Nat Cancer 2020; 1:1153-66
30. Wang H, et al. Nat Immunol 2020; 21:298-308
31. Schapira M, et al. Nat Rev Drug Discov 2019; 18:949-63
32. Li K, et al. Bioinformatics 2019; 35:1249-51
33. Kotecha N, et al. Curr Protoc Cytom 2010; Chapter 10:Unit10 7
34. Chen H, et al. PLOS Comput Biol 2016; 12:e1005112
35. Butler A, et al. Nat Biotechnol 2018; 36:411-20
36. Zhang L, et al. Nature 2018; 564:268-72
37. Li T, et al. Nucleic Acids Res 2020; 48:W509-W14
38. Chen B, et al. Methods Mol Biol 2018; 1711:243-59
39. Finotello F, et al. Genome Med 2019; 11:34
40. Aran D, et al. Genome Biol 2017; 18:220
41. Li B, et al. Genome Biol 2016; 17:174
42. Racle J, et al. Elife 2017; 6

### Example 2: Enhancing PD-L1 Degradation by ITCH Improves Response to Immune Checkpoint Blockade

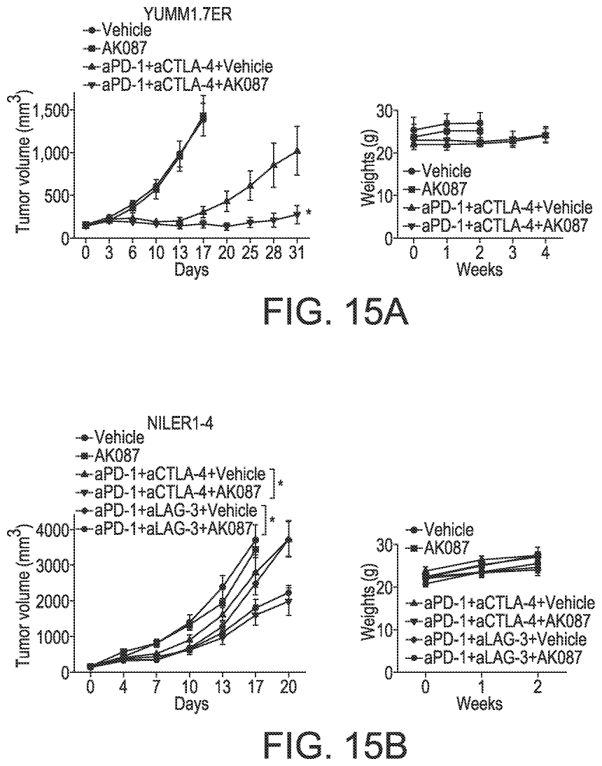

As demonstrated in FIGS. 15A-15B, the ITCH agonist AK087 improves responses of melanoma to immune checkpoint blockade. Data are derived from two murine models of melanoma, YUMM1.ER, Braf^{V600MUT} melanoma, and NILER1-4, Nras^{Q60MUT} melanoma. FIG. 15A shows results from YUMM1.7ER, with n=9-10 per group. Means±SEM. Anti-PD-1 200 μg/mouse, anti-CTLA-4 200 μg/mouse three times a week for the first two weeks, then changed to twice/week treatment. AK087 10 mg/kg/day (Subcutaneously, daily) administration. *p<0.05, student's t test. FIG. 15B shows results from NILER1-4, with n=8-10 per group. Means±SEM. Anti-PD-1 200 μg/mouse, anti-CTLA-4 200 μg/mouse, aLAG-3 200 μg/mouse three times a week for the first two weeks, then changed to twice/week treatment. AK087 15 mg/kg/day (Subcutaneously, daily) administration. *p<0.05, student's t test.

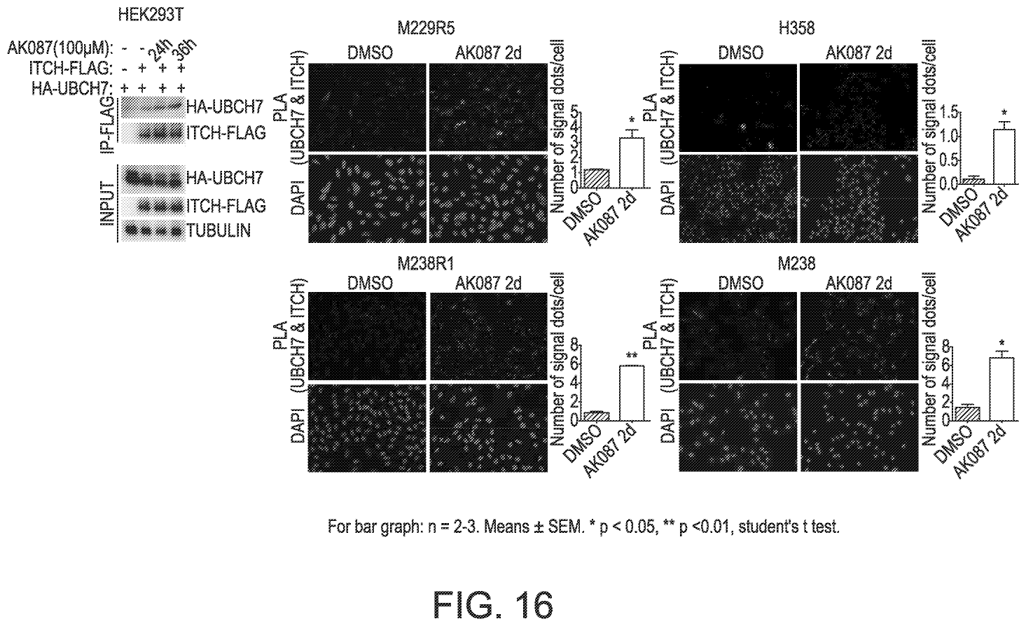

FIG. 16 demonstrates that the ITCH agonist AK087 enhances the interaction between UBCH7 and ITCH in HEK293T cells (left panel) and cancer cell lines M229R5, H358, M238R1 and M238 (right panel), which is the mechanism by which AK087 enhances ITCH activity. For bar graph: n=2-3. Means #SEM. *p<0.05, **p<0.01, student's t test.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

| Com- | | Screening | | | | |
|---|---|---|---|---|---|---|
| pound | | concentration | Activity | Vendor & | | |
| Number | Structure | (uM) | (%) | Supplier ID | Formula | Name |
| RL 1 | 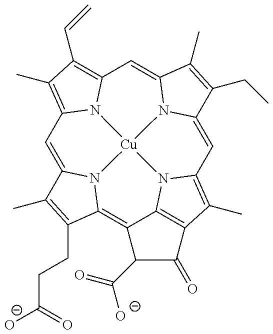 | 20 | 147 | NINDS (CHLORO- PHYLLIDE Cu COMPLEX Na SALT. Cas no. 15611-43-5) 1505308 | C34H28CuN4Na2O5-4 | CHLORO- PHYLLIDE Cu COMPLEX Na SALT |
| RL 2 | 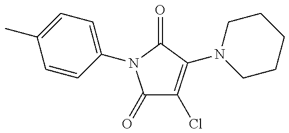 [SMILES: Cc1ccc(N2C(=O)C(Cl)=C(N3CCCCC3)C2=O)cc1] | 10 | 141 | SPECS specs.net AF-399/14183590 | C16H17ClN2O2 | 3-chloro-1-(4- methylphenyl)- 4- piperidin-1-yl- 1H-pyrrole-2,5- dione |
| RL 3 | 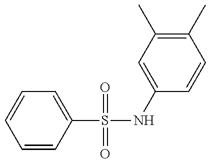 [SMILES: Cc1ccc(NS(=O)(=O)c2ccccc2)cc1C] | 10 | 140 | SPECS specs.net AN-652/11634463 | C14H15NO2S | N-(3,4- dimethyl- phenyl) benzene- sulfonamide |
| RL 4 | 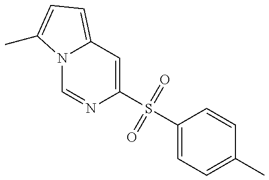 | 10 | 145 | SPECS specs.net AQ-405/42300255 | C15H14N2O2S | 4-methyl- phenyl 7-methyl- pyrrolo[1,2-c] pyrimidin-3-yl sulfone |
| RL 5 | 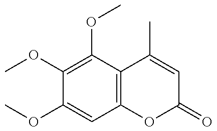 [SMILES: COc1cc2oc(=O)cc(C)c2c(OC)c1OC] | 10 | 140 | SPECS specs.net AK-087/33381003 | C13H14O5 | 5,6,7- trimethoxy-4- methyl-2H- chromen-2- one |
| RL 6 | 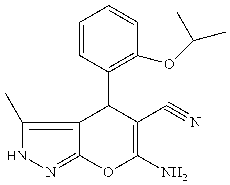 [SMILES: Cc1[nH]nc2c1C(c1ccccc1OC(C)C)C(C#N)=C(N)O2] | 10 | 194 | SPECS specs.net AM-807/14961541 | C17H18N4O2 | 6-amino-3- methyl-4-{2- [(1- methylethyl) oxy] phenyl}-2,4- dihydro- pyrano[2,3-c] pyrazole-5- carbonitrile |

1. A method of suppressing tumor-surface PD-L1 expression to thereby enhance anti-cancer therapy in a subject in need thereof, the method comprising administering to the subject an effective amount of a modulator of tumor cell-surface PD-L1/L2.

2. The method of claim 1, wherein the modulator of tumor cell-surface PD-L1/L2 is an activator of E3 ligase ITCH and/or a destabilizer of tumor cell-surface PD-L1/L2.

3. The method of claim 2, wherein the activator of E3 ligase ITCH is selected from: Chlorophyllide, 3-chloro-1-(4-methylphenyl)-4-piperidin-1-yl-1H-pyrrole-2,5-dione, N-(3,4-dimethylphenyl)benzenesulfonamide, 4-methylphenyl 7-methylpyrrolo[1,2-c]pyrimidin-3-yl sulfone, 5,6,7-trimethoxy-4-methyl-2H-chromen-2-one (AK087), and 6-amino-3-methyl-4-{2-[(1-methylethyl)oxy]phenyl}-2,4-dihydropyrano[2,3-c]pyrazole-5-carbonitrile.

4. The method of claim 1, wherein the destabilizer of tumor cell-surface PD-L1/L2 is a recombinant bispecific antibody-based proteolysis-targeting chimera (AbTAC) that recruits membrane-bound E3 ligases to degrade cell-surface PD-L1/L2.

5. The method of claim 1, wherein the subject is treated with one or more mitogen-activated protein kinase (MAPK) inhibitors, and optionally, one or more immune checkpoint antibodies as anti-cancer therapy.

6. The method of claim 5, wherein the MAPK inhibitor(s), or MAPK inhibitor(s) and one or more immune checkpoint antibodies, is administered concomitantly with, prior to, and/or subsequent to the administering of the activator of ITCH or destabilizer of cell-surface PD-L1/L2.

7. The method of claim 5, wherein the MAPK inhibitor is selected from: Vemurafenib, Dabrafenib, Encorafenib, Trametinib, Binimetinib, and Cobimetinib, as well as type II RAF inhibitors or pan-RAF inhibitors, such as BGB-283, BGB-3245, DAY101/TAK-580, KIN-2787, and LXH254.

8. The method of claim 1, wherein the subject is treated with anti-CTLA-4 antibody, anti-PD-1 antibody, anti-PD-L1 antibody, and/or anti-LAG-3 antibodies as anti-cancer therapy.

9. A method of inhibiting an adaptive immune resistance response to immune checkpoint therapy in a subject in need thereof, the method comprising administering to the subject an effective amount of an activator of E3 ligase ITCH or a destabilizer of cell-surface PD-L1/L2.

10. The method of claim 9, wherein the immune checkpoint therapy comprises anti-CTLA-4 antibody, anti-PD-1 antibody, anti-PD-L1 antibody, and/or anti-LAG-3 antibody treatment.

11. The method of claim 1, wherein the modulator of tumor cell-surface PD-L1/L2 is 5,6,7-trimethoxy-4-methyl-2H-chromen-2-one (AK087).

12. The method of claim 1, wherein the subject is in need of treatment for melanoma, pancreatic ductal adenocarcinoma, or colorectal adenocarcinoma.

13. The method of claim 9, wherein the modulator of tumor cell-surface PD-L1/L2 is 5,6,7-trimethoxy-4-methyl-2H-chromen-2-one (AK087).

14. The method of claim 9, wherein the subject is in need of treatment for melanoma, pancreatic ductal adenocarcinoma, or colorectal adenocarcinoma.
